(19) United States
(12) Patent Application Publication
(10) Pub. No.: US 2016/0166208 A1
(43) Pub. Date: Jun. 16, 2016

(54) Method and Apparatus for Detecting and Classifying Seizures

(71) Applicant: Brain Sentinel, Inc., San Antonio, TX (US)

(72) Inventors: Michael R. Girouard, San Antonio, TX (US); Russell M. Herring, San Antonio, TX (US); Jose E. Cavazos, San Antonio, TX (US)

(21) Appl. No.: 14/920,665

(22) Filed: Oct. 22, 2015

Related U.S. Application Data
(63) Continuation-in-part of application No. PCT/US14/68246, filed on Dec. 2, 2014.
(60) Provisional application No. 62/050,054, filed on Sep. 12, 2014.
(60) Provisional application No. 62/032,147, filed on Aug. 1, 2014.
(60) Provisional application No. 62/001,302, filed on May 21, 2014.
(60) Provisional application No. 61/979,225, filed on Apr. 14, 2014.
(60) Provisional application No. 61/969,660, filed on Mar. 24, 2014.
(60) Provisional application No. 61/910,827, filed on Dec. 2, 2013.

(51) Int. Cl. A61B 5/00; A61B 5/0488

(52) U.S. Cl. CPC A61B 5/7264; A61B 5/0488; A61B 5/7282; A61B 5/746; A61B 5/4094

(57) ABSTRACT

A method of monitoring a patient for seizure activity may include monitoring the patient by collecting an EMG signal and determining whether samples of signal that include regions of elevated signal amplitude are present in the collected signal. The samples may further be qualified based on one or more properties of a clonic-phase portion of a seizure, and if the samples are qualified, a qualified-clonic-phase burst activity level may be determined. A response may then be executed if the qualified-clonic-phase burst activity level exceeds a threshold value. Related apparatuses are also described.

### CROSS-REFERENCE TO RELATED APPLICATION

This application is a continuation-in-part of PCT International Application No. PCT/US14/68246 filed Dec. 2, 2014, which claims priority to U.S. Provisional Patent Application No. 61/910,827 filed Dec. 2, 2013, U.S. Provisional Patent Application No. 61/969,660 filed Mar. 24, 2014, U.S. Provisional Patent Application No. 61/979,225 filed Apr. 14, 2014, U.S. Provisional Patent Application No. 62/001,302 filed May 21, 2014, U.S. Provisional Patent Application No. 62/032,147 filed Aug. 1, 2014, and U.S. Provisional Patent Application No. 62/050,054 filed Sep. 12, 2014. This application also claims priority to U.S. Provisional Patent Application No. 62/096,331 filed Dec. 23, 2014. The disclosures of all of the foregoing applications are herein fully incorporated by reference.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system activating different muscles of the body.

Techniques designed for studying and monitoring seizures have typically relied upon electroencephalography (EEG), which characterizes electrical signals using electrodes attached to the scalp or head region of a seizure prone individual or seizure patient. In EEG, electrodes may be positioned so as to measure such activity; that is, electrical activity originating from neuronal tissue. Alternatively, electromyography (EMG) may be used for seizure detection. In EMG, an electrode may be placed on or near the skin, over a muscle, to detect electrical activity resulting from muscle fiber activation.

Detecting an epileptic seizure using EEG typically requires attaching many electrodes and associated wires to the head and using amplifiers to monitor brainwave activity. The multiple EEG electrodes may be very cumbersome and generally require some technical expertise to apply and monitor. Furthermore, confirming a seizure requires observation in an environment provided with video monitors and video recording equipment. Unless used in a staffed clinical environment, such equipment may not be intended to determine if a seizure is in progress, but rather provide a historical record of the seizure after the incident. Such equipment is usually meant for hospital-like environments where a video camera recording or caregiver's observation may provide corroboration of the seizure, and is typically used as part of a more intensive care regimen such as a hospital stay for patients who experience multiple seizures. Upon discharge from the hospital, a patient may be sent home, often with little further monitoring.

Ambulatory devices for diagnosis of seizures are generally EEG-based, but because of the above shortcomings those devices are not designed or suitable for long-term home use or daily wearability. Other seizure alerting systems may operate by detecting motion of the body, usually the extremities. Such systems may generally operate on the assumption that while suffering a seizure, a person will move erratically and violently. For example, accelerometers may be used to detect violent extremity movements. However, depending upon the type of seizure, this assumption may or may not be true. Electrical signals sent from the brain during some seizures may be transmitted to many muscles simultaneously, which may result in muscles fighting each other and effectively canceling out violent movement. In other words, the muscles may work to make the person rigid rather than cause actual violent movement. Thus, some seizures may not be consistently detected with accelerometer-based detectors.

Ambulatory devices for diagnosis of seizures are generally not suited to grade seizures based on intensity, nor are they suited to differentiate seizure-related signals based on event type. Rather, different types of seizures may often be grouped together. Accordingly, ambulatory devices for seizure detection may be ill-suited to customize responses for different types of detected seizure events. However, not all seizures or seizure-related events may necessarily demand the same response. For example, at least for some patients or some patients in certain situations, seizure events may be detected and the event recorded, but without automatic initiation of a complete and costly emergency response. Thus, other ambulatory devices are not ideally suited for cost-effective monitoring of some patients. Also, using current ambulatory devices, caregivers may misdiagnose some conditions, including, some that may benefit from condition-specific therapies. For example, some events, such as psychogenic or non-epileptic seizure events, may be grouped together with generalized tonic-clonic seizure events. Statistical analysis of event signals may encourage effective diagnosis of some commonly misdiagnosed conditions. However, other ambulatory detection systems are generally not configured to provide organized statistical information to caregivers as may be used to medically or surgically manage a patient's care.

Accordingly, there is a need for epileptic seizure detection methods and apparatuses that can be used in non-institutional or institutional environments without many of the cumbersome electrodes to the head or extremities. There is further a need for detection methods that are suited to grade seizures by type and/or intensity and customize alarms so as to provide robust and cost effective patient care. There is also a need for monitoring systems that organize medical data within databases to help medically and surgically manage patient care.

### SUMMARY

A method of monitoring a patient for seizure activity may include detection of samples of EMG signals that may include short-lived regions of amplitude elevation. Those samples may further be compared to one or more thresholds in qualification protocols suitable to identify that the samples may be associated with the clonic phase of a seizure. In some embodiments, samples may further be organized into one or more groups to facilitate accurate qualification of samples and to identify activity associated with the clonic phase of a seizure. Qualified samples may be used to determine the presence of a seizure and to classify particular parts of seizure activity. Routines for selective detection of the clonic phase of a seizure may further be used with other detection routines configured for detection of tonic-phase seizure activity and/or with routines configured for detection of motor manifestations that may indicate elevated risk that a patient may be progressing towards a seizure state. Methods incorporating those routines may be used to provide cost effective monitoring strategies where responses may be tailored to specific detected signal activity. A statistical analysis of individual samples of signals may further be communicated to caregivers and used to help caregivers manage a patient's care. For example, trends in qualified sample activity throughout the course of a seizure, including, for example, during seizure recovery, may be used to assist caregivers with identification of patient conditions that may commonly be confused with epileptic seizures. Apparatuses suitable to execute methods herein are further described.

In some embodiments, a method of monitoring a patient for seizure activity may include monitoring a patient by collecting an EMG signal using one or more EMG electrodes. Methods may further include processing of the collected EMG signal to identify if one or more samples of the EMG signal that include an elevation in signal amplitude are present and determining if the one or more samples meet one or more qualification thresholds suitable to identify that the one or more samples are indicative of clonic-phase seizure activity. In some embodiments, the one or more qualification thresholds may include one or more duration width thresholds selected from a group of duration width thresholds including a maximum duration width threshold, a minimum duration width threshold, and a combination of both a maximum duration width threshold and a minimum duration width threshold. Samples that meet the qualification thresholds may be used to determine a qualified-clonic-phase burst activity level and to determine if a seizure is present.

### BRIEF DESCRIPTION OF THE DRAWINGS

FIG. 1 illustrates an embodiment of a method for monitoring a patient for seizure-related activity.

FIG. 2 illustrates another embodiment of a method for monitoring a patient for seizure-related activity.

FIG. 3 illustrates a part of a routine that may be used in a method of monitoring a patient for seizure-related activity.

FIG. 4 illustrates another part of a routine that may be used in a method of monitoring a patient for seizure-related activity.

FIG. 5 illustrates still another part of a routine that may be used in a method of monitoring a patient for seizure-related activity.

FIG. 6 illustrates an embodiment of a method for identifying parts of an EMG signal.

FIG. 7 illustrates another embodiment of a method for identifying parts of an EMG signal.

FIG. 8 illustrates an embodiment of a method for grouping or organizing samples of EMG signal into a sample train or group.

FIG. 9 illustrates an embodiment of a routine for analyzing the periodicity of data.

FIG. 10 illustrates an embodiment of a system for monitoring a patient for seizure activity.

FIG. 11 illustrates an embodiment of a detection unit.

FIG. 12 illustrates an embodiment of a base station.

FIG. 13 illustrates exemplary EMG signal data for a patient.

FIG. 14 illustrates EMG signal data collected during the clonic phase of a seizure.

FIG. 15 illustrates exemplary EMG signal data for an isolated band of signals between 30 Hz and 40 Hz.

FIG. 16 illustrates exemplary EMG signal data for an isolated band of signals between 75 Hz and 85 Hz.

FIG. 17 illustrates exemplary EMG signal data for an isolated band of signals between 130 Hz and 240 Hz.

FIG. 18 illustrates exemplary EMG signal data for an isolated band of signals between 300 Hz and 400 Hz.

FIG. 19A illustrates further EMG signal data collected during the clonic phase of a seizure.

FIG. 19B shows a model clonic-phase-burst pattern of the data in FIG. 19A.

FIG. 20A illustrates still further EMG signal data collected during the clonic phase of a seizure.

FIG. 20B shows a model clonic-phase-burst pattern of the data in FIG. 20A.

### DETAILED DESCRIPTION

The following terms as used herein should be understood to have the indicated meanings.

When an item is introduced by “a” or “an,” it should be understood to mean one or more of that item.

“Comprises” means includes but is not limited to.

“Comprising” means including but not limited to.

“Computer” means any programmable machine capable of executing machine-readable instructions. A computer may include but is not limited to a general purpose computer, microprocessor, computer server, digital signal processor, or a combination thereof. A computer may comprise one or more processors, which may comprise part of a single machine or multiple machines.

The term “computer program” means a list of instructions that may be executed by a computer to cause the computer to operate in a desired manner.

The term “computer readable medium” means an article of manufacture having a capacity for storing one or more computer programs, one or more pieces of data, or a combination thereof. A computer readable medium may include but is not limited to a computer memory, hard disk, memory stick, magnetic tape, floppy disk, optical disk (such as a CD or DVD), zip drive, or combination thereof.

“Having” means including but not limited to.

“Routine” refers to a method or part of a method that may be used to monitor a patient for seizure activity. A routine may be run individually in a strategy for monitoring a patient or may be run in combination with other routines or methods in an overall strategy for patient monitoring.

Where a range of values is described, it should be understood that intervening values, unless the context clearly dictates otherwise, between the upper and lower limit of that range and any other stated or intervening value in other stated ranges, may be used within embodiments herein.

The apparatuses and methods described herein may be used to detect seizures and timely alert caregivers of seizure-related events. The apparatuses may include sensors disposed on, near, or underneath the skin of a patient or attached to a patient's clothing and may be configured for measurement of muscle electrical activity using EMG. In some embodiments, apparatuses herein may include one or more processors suitable to receive an EMG signal and process the information to detect seizure-related signals. Detection of seizures using EMG electrodes is further described in, for example, Applicant's U.S. Pat. No. 8,983,591, Applicant's U.S. patent application Ser. Nos. 13/542,596 and 14/816,924, Applicant's International Applications PCT/US14/61783 and PCT/US14/68246, and Applicant's U.S. Provisional Patent Application Nos. 61/875,429, 61/894,793, 61/910,827, 61/969,660, 61/979,225, 62/001,302, 62/032,147, 62/050,054, and 62/096,331, the disclosures of each of which are herein fully incorporated by reference. As described in this disclosure, apparatuses and methods may be used to monitor a patient for muscle electrical activity by collecting EMG signals and may include one or more routines that may evaluate collected EMG signals for activity associated with different parts of a seizure. For example, some routines may be responsive to motor manifestations that may be present near the start of a seizure or during the tonic phase of a seizure. Other routines may be selective for detection of the clonic phase of a seizure. In some embodiments, a seizure's progression through different seizure phases and/or seizure parts may be identified. For example, patterns of transition between early portions of the clonic phase of a seizure and later portions of the clonic phase of a seizure may be identified and provided to a caregiver to help manage a patient's care.

In some embodiments, a response that may be executed based on a detected seizure or seizure-related event may be tailored based upon characteristics of the detected event. For example, some responses may include initiation of one or more particular warning or emergency alarm protocols. Characteristics of detected events, which may be used to determine a response, may, for example, include the event type, which may include, for example, tonic-clonic, tonic-only, clonic-only, or other types of seizure or seizure-related events. In some embodiments, detected events may also be characterized based on an event intensity or graded strength. In some embodiments, apparatuses and methods herein may further be used to create a searchable log of seizure events to help medically or surgically manage a patient. To facilitate organization of detected seizure or seizure-related events, some events may be automatically classified. For example, automatic classification of seizure events (e.g., based on type and/or graded severity) may be used in the creation of ordered databases including seizure-related data, which is a particularly valuable feature where video corroboration of events is absent or where individual review of sizeable amounts of data by trained professionals, such as medical doctors, would be inconvenient or prohibitively costly.

In some embodiments, methods of monitoring a patient for seizure activity may include one or more routines configured for collection and/or analysis of an EMG signal for characteristics of seizure activity, the detection of which may not only indicate the presence of a seizure, but also may indicate detection of a given part of a seizure, such as the tonic or clonic phase of a seizure. For example, a characteristic of a collected EMG signal (or routine to measure the characteristic) may be “selective” for a part of a seizure, and the characteristic may be detected in a patient experiencing that seizure part, but the characteristic may be substantially absent, undetectable, or give a substantially different output when that part of a seizure is absent.

In some embodiments, routines configured for selective detection of the clonic phase of a seizure may include one or more steps to identify samples of EMG signal that may include an elevated region or portion of signal over a background or reference level. In some embodiments, an identified sample may include a portion of elevated signal over background and an adjacent portion of reduced signal amplitude in comparison to the portion with elevated signal amplitude. The elevated portion of a sample may be referred to as a peak, and in some embodiments, a sample may be detected using one or more peak detection algorithms. For example, some of the peak detection algorithms that may be used in methods herein may detect peaks by identifying one or more peak edges, including, for example, a leading edge of a peak, a trailing edge of a peak, and/or both leading and trailing edges of a peak.

In some embodiments, samples may be qualified to identify that the samples may properly be associated with the clonic phase of a seizure. For example, qualification may include determining one or more values for one or more properties of a sample or group of samples. Determined property values may further be compared to one or more qualification thresholds. For example, in some embodiments, if a property value of a sample meets a qualification threshold, the sample may be deemed to be qualified and may be referred to as a qualified-clonic-phase burst.

In some embodiments, qualification thresholds may be configured to identify samples that are indicative of clonic-phase activity and to discriminate clonic-phase activity from other seizure and/or non-seizure activity. For example, samples of signal that pass qualification, which may then be classified as qualified-clonic-phase bursts, may be discriminated from other signals that may also include amplitude elevations, including, for example, signals resulting from tonic-phase activity which, when using certain thresholds as described herein, may not pass qualification. In some embodiments, samples of an EMG signal may be qualified in one or more stages or steps in which properties of samples are compared to thresholds in order to determine whether they may be associated with clonic-phase activity. In this disclosure, where a sample of an EMG signal is subject to an intermediate stage in overall qualification, the sample may, if it meets the intermediate stage in qualification, also be referred to as a prequalified sample. In some embodiments, a clonic-phase burst activity level may be determined based on the detection of qualified-clonic-phase bursts. For example, a clonic-phase burst activity level may include a burst count number, burst count rate, certainty-weighted burst value, other metric of burst activity, and combinations thereof. An activity level may then be used to determine if the clonic phase of a seizure is detected.

In some embodiments, qualification of data may include a comparison of one or more properties of individual samples of signal to one or more qualification thresholds. In other embodiments, qualification of data may include grouping of more than one sample together, with qualification accomplished by comparing an aggregate property of a group of samples to an aggregate property threshold. Still in other embodiments, qualification of data may include a combination of steps including individual sample qualification and also qualification of sample groups. A value of a property of a sample or of a sample group may be referred to as a “property value.” A value of a property that is specifically associated with a sample group may also be referred to as an “aggregate property value.” A qualification threshold value related to a property of a group of samples may likewise be referred to as an “aggregate qualification threshold value.” For example, included among aggregate qualification threshold values that may be used to qualify samples in a group are one or more of a minimum deviation value calculated from duration widths of samples or parts of samples, a maximum deviation value calculated from duration widths of samples or parts of samples, a minimum rate of sample repetition, a maximum rate of sample repetition, a minimum regularity of one or more sample characteristics, a maximum regularity of one or more sample characteristics, and/or combinations of the aggregate qualification threshold values thereof.

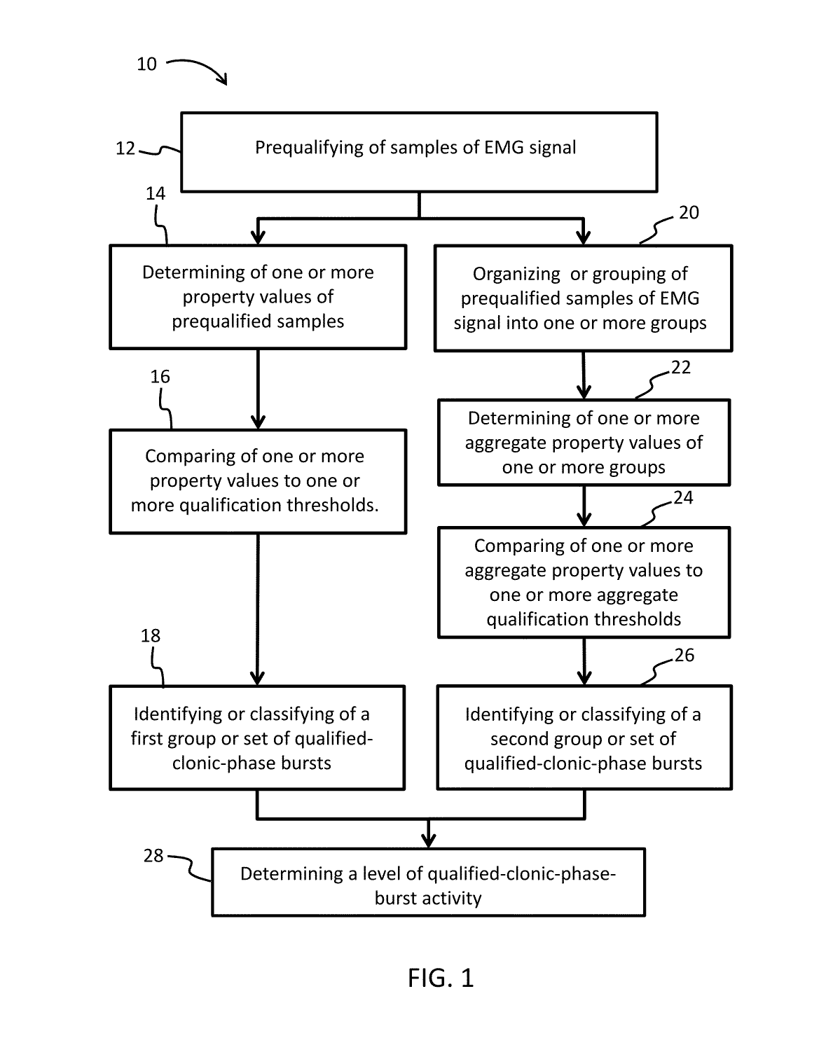

FIG. 1 illustrates embodiments of a method 10 for monitoring a patient for seizure activity that may include one or more qualification steps. In some of those embodiments, samples of an EMG signal may be prequalified and then further qualified by comparison of either or both of property values of individual samples and/or property values of sample groups to associated qualification threshold values. For example, in a step 12, samples of an EMG signal may be detected and prequalified. The prequalified samples may then be qualified using remaining steps in either or both of two qualification paths. In some embodiments, the qualifications paths may be executed together. Alternatively, the two qualification paths may be executed separately as individual routines or methods used for patient monitoring.

In a first qualification path, including steps 12, 14, 16, and 18, a first group of qualified-clonic-phase bursts may be identified. In step 14, one or more property values of prequalified samples identified in step 12 may be determined. In step 16, the one or more property values may be compared to property value thresholds. Based on that comparison, as shown in step 18, the first group or set of qualified-clonic-phase bursts may be identified. In a second qualification path, including steps 12, 20, 22, 24, and 26, a second group of qualified-clonic-phase bursts may be identified. In step 20, prequalified samples, including those identified in step 12, may be organized into one or more groups. In step 22, one or more aggregate property values of the one or more groups may be determined. In step 24, the one or more aggregate property values may be compared to one or more aggregate qualification thresholds. Based on that comparison, as shown in step 26, the second group or set of qualified-clonic-phase bursts may be identified. In step 28, an overall level of qualified-clonic-phase-burst activity may be determined. For example, in some embodiments, the first group of qualified-clonic-phase bursts and the second group of qualified-clonic-phase bursts may be combined to determine an overall activity level such as a burst count. An activity level may then be used to evaluate whether a clonic-phase portion of a seizure is present.

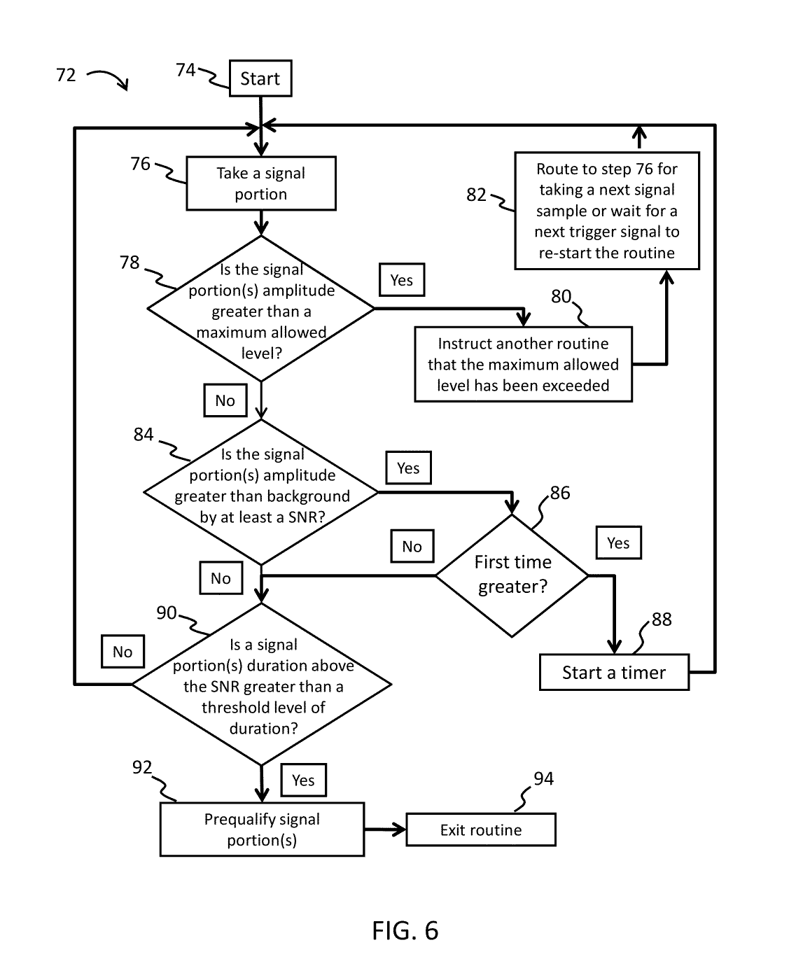

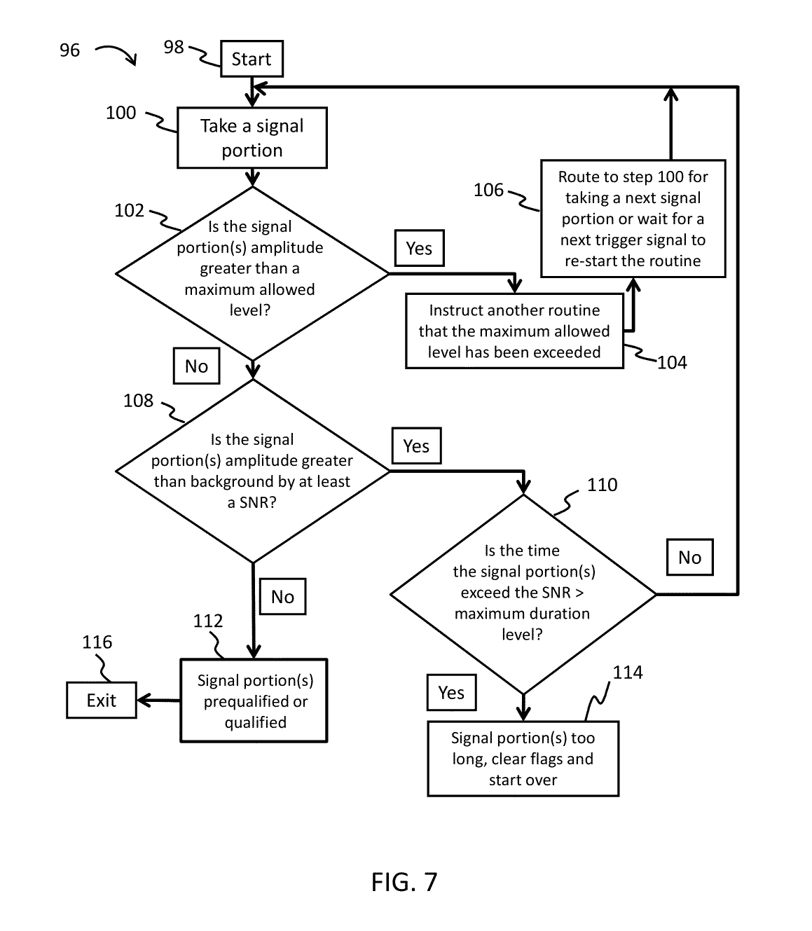

In step 12, prequalifying of samples may be based on various sample properties and threshold conditions that may be met by a given sample of signal included in a collected signal. For example, in some embodiments, as also explained in detail in a routine 72, illustrated in FIG. 6, and a routine 96, illustrated in FIG. 7, an EMG signal may be collected in step 12 and elevated portions of the signal or samples may be detected. The samples may be prequalified based on meeting a threshold condition of including a region of sufficient elevated signal amplitude over background or noise. The routines 72 and 96 further describe prequalifying of samples based on whether samples meet other threshold conditions, including, for example, either or both of a minimum duration width and/or a maximum duration width. The routines 72 and 96 may be executed individually or in combination, and prequalification of samples in step 12 may include prequalifying of samples based on either or both of those routines. Accordingly, in some embodiments herein, prequalification of samples may be based on combinations of threshold conditions as described in the routines 72 and 96.

The first path for qualification of samples may include steps 12, 14, 16, and 18. In step 14, one or more property values may be determined for one or more properties of the prequalified samples identified in step 12. In step 16, property values may then be compared to qualification thresholds. Particularly, in the first path of method 10, properties and thresholds may be associated with individual samples. For example, in some embodiments, qualification may include comparison of values of properties for individual samples or parts of individual samples to qualification thresholds, including, for example, minimum duration width, maximum duration width, minimum signal-to-noise, maximum amplitude, maximum duration of an elevated portion of a sample, minimum duration of an elevated portion of a sample, minimum duration of an adjacent quiet period of signal, maximum duration of an adjacent quiet period of signal, other suitable qualification thresholds, and combinations thereof.

In some embodiments, one or more similar or equivalent properties of samples may be used to identify or prequalify samples in step 12 and to qualify samples in steps 14 and 16. For example, in some embodiments, prequalifying step 12 and qualifying steps 14 and 16 may each include comparison of a duration width value to one or more duration width thresholds. In some embodiments, steps 12, 14, and 16 may be performed concurrently or consecutively, including, for example, as one set of instructions executed by a processor. Thus, operations described in step 12 and qualification described in steps 14 and 16 may be executed together as one processing step. However, in some embodiments, it may be useful to apply different thresholds and/or a different set of properties for prequalification and qualification of samples. For example, one set of properties and thresholds may be applied to identify or prequalify samples in step 12, but other properties and thresholds may be used to qualify samples in steps 14 and 16. More generally, in some embodiments, a first group of properties and/or thresholds may be applied in step 12, and a second group of properties and/or thresholds may be applied in steps 14 and 16. In some embodiments, it may be useful to apply one or more processing operations such as smoothing, filtering, or peak fitting between or as part of one or more prequalification and/or one or more qualification steps. For example, in some embodiments, a first routine may include executing processing instructions to rapidly identify and prequalify samples based on instructions that may be rapidly executed by a processor. For example, in some embodiments, prequalifying may include determining signal amplitude values that may be compared to a pre-set baseline reference value without needing to perform certain curve fitting or smoothing operations on a collected signal. However, when qualifying samples, the aforementioned one or more processing operations or other suitable operations may be used to more accurately measure values of certain properties so that a more robust comparison to threshold values may be made. In some embodiments, as also described in reference to FIG. 6, peak fitting operations that may be used to define one or more peak edges may be executed together with prequalification or after prequalification.

As shown in step 18, a first group of qualified-clonic-phase bursts may be identified or classified. For example, in some embodiments, prequalified samples identified in step 12 that meet the qualification conditions in steps 14 and 16 may be included, as shown in step 18, in the first group of qualified-clonic-phase bursts.

A second path for qualification of samples may include steps 12, 20, 22, 24, and 26. In step 20, prequalified samples of EMG signal data, including, for example, samples identified in step 12, may be organized into one or more groups. For example, in some embodiments, organizing of prequalified samples in step 20 may include grouping together prequalified samples present within a certain time period. For example, in some embodiments, when prequalified samples are identified during execution of step 12, the prequalified samples may be placed into a circular data buffer. That data buffer may be periodically scanned using a managing processor or algorithm. In some embodiments, each prequalified sample identified upon scanning the buffer may be included in a train or group of samples. As referred to herein, a train of samples, such as a train of prequalified samples, is a group of samples or prequalified samples adjacent in time.

In some embodiments, a buffer may be scanned periodically at regular intervals. Accordingly, a train or group of samples may include prequalified samples identified over some regular or selected time period. For example, in some embodiments, a buffer may be scanned about every 2 seconds to about every 5 seconds.

In some embodiments, prequalified samples may be grouped in step 20 by placing prequalified samples into a circular data buffer that may be scanned based on a trigger or initiation signal. For example, in some embodiments, if some selected number of prequalified samples is added to a buffer, a scan of the buffer may be initiated. Accordingly, a group of prequalified samples or a train may include a selected number of samples such as about 3 samples to about 20 samples. In some embodiments, if one or more properties of prequalified samples changes by a certain level, scanning and analysis of a train may be initiated. For example, one part of a processor, such as a register, may store information about the duration width of prequalified samples, and if duration width values change by some amount, scanning of a buffer for a train and analysis of the train may be initiated. For example, in some embodiments, for each prequalified sample, the duration width of adjacent periods of reduced signal adjacent to elevated portions of a prequalified sample may be determined and stored in a data register, and if the duration width changes by some amount, scanning of a buffer for a train of prequalified samples may be initiated.

In some embodiments, it may be valuable to group together, as indicated in step 20, all prequalified samples identified in step 12 that are present over some time period. Accordingly, all prequalified samples may be subject to aggregate qualification testing as described in steps 24 and 26. In other embodiments, a subset of a set of prequalified samples identified in step 12 may be removed before being organized or grouped in step 20. For example, in some embodiments, prequalified samples may be identified in step 12 based on one or more duration width thresholds. Those prequalified samples may be further processed by removing samples from consideration if elevated portions of the samples are too close together or too far apart. The resulting filtered set of samples may then be organized or grouped together as shown in step 20.

In some embodiments, step 20 may include a prequalification sub-step that may be executed following the prequalification step 12. For example, in some embodiments, one or more prequalified samples identified in step 12 may be removed based on a comparison of a duration width of an adjacent quiet period of a sample to one or more duration width thresholds. Remaining samples meeting that additional prequalification condition may then be organized in remaining parts of step 20 and included in one or more sample groups. Adding a prequalification condition that elevated portions of samples may not be too close together or too far apart or adding a prequalification condition of a minimum duration width of an adjacent quiet period of a sample may help avoid noise spikes or other artificial signatures from inadvertent inclusion and skewing of group sample data. Therefore, in some embodiments herein, it should be understood that prequalification conditions used to generate an input set of samples for qualification in the first path of method 10 and prequalification conditions used to generate another input set of samples for qualification in the second path of method 10 may be the same or different.

In some embodiments, prequalified samples may be excluded from groups that are made or constructed in he step 20 based on one or more other considerations. For example, in some embodiments, organization of prequalified samples in step 20 may include sequential removal of one or more prequalified samples from a larger group of prequalified samples as may be used to construct test groups including various numbers of included prequalified samples. In some embodiments, prequalified samples may be excluded from one or more groups based on other criteria. For example, a procedure may order samples based on one or more peak characteristics. Based on that ordering, a list of candidate samples including peaks most likely to be spurious (e.g., not related to physiological activity) may be generated. For example, a group of samples may be ordered based on peak or elevated sample portion duration width, and that ordering may be used to identify that the duration width of a majority of samples are clustered around a central range, but one or more samples may be characterized as having an elevated portion with a duration width outside of that central range. Samples outside of the most common range may be removed or first removed in generation of test groupings. For example, in some embodiments, prequalified samples may be excluded from some groups as part of routines configured, for example, to search for particular patterns of activity. Removal of prequalified samples and test group construction, including as related to identification of patterns in data that may be noisy or intermittent, is also described in greater detail in Applicant's U.S. Provisional Application No. 62/096,331, the disclosure of which is incorporated herein by reference.

In some embodiments, samples may be grouped in sample trains that may extend for a suitable duration such that a train may be associated with a distinct or limited part of the clonic phase of a seizure. For example, in some embodiments, as further described in Example 4, a train or group of samples may include samples present in time periods of between about 2 seconds to about 5 seconds. In some embodiments, grouping of samples by collecting samples in time periods that are too short may result in collection of too small a number of samples to be reliably qualified based on aggregate or group properties. For example, in some embodiments, grouping of too few a number of samples may negatively affect qualifying in steps 22 and 24 because an insufficient number of samples may be included in a group to reliably determine some aggregate property values. However, grouping of samples by collecting samples in time periods that are too great a time period may yield a train that extends throughout all or a significant proportion of a seizure's clonic phase. And, including samples present throughout too great a proportion of a seizure's clonic phase together in a group may negatively impact some calculations that involve comparing aggregate qualification property values to thresholds as described in the qualifying steps 22 and 24. For example, groups of samples that extend across too great a duration of the clonic phase may sometimes have less predictable aggregate property values, including, for example, property values related to the average or spread of duration widths of periods between samples, than some other groups that extend over a shorter duration.

For example, duration widths of periods between elevated parts of samples, as well as overall sample duration width if an adjacent region quiet period is included along with a peak portion of a sample, may increase throughout the course of the clonic phase, particularly during later parts of the clonic phase of a seizure. Accordingly, calculations associated with the average or spread of periods of times between samples may depend upon how many samples are grouped together or the time frame over which a group of samples persists. Groups of more limited sample number, which persist over a more limited duration, may generally show a variation of duration widths of periods between samples that is associated with natural variation in how physiological events associated with the clonic phase are manifested. Groups including greater numbers of samples, which persist over a longer duration, may show a variation of duration widths of periods between samples that is not only related to how physiological events naturally vary in any given part of the clonic phase, but also related to how periods between samples change over time, including, for example, during extended portions of the clonic phase and particularly seizure recovery. Accordingly, some embodiments herein may construct groups of suitable duration, such as, for example, over time periods of about 2 seconds to about 5 seconds, to encourage accurate qualification of seizure-related samples thereby improving detection performance. An additional advantage of some of those embodiments is that statistics for samples in adjacent groups may be reliably determined and trends in sample statistics tracked over time between adjacent groups or trains. In addition, some of the embodiments herein that group samples based on how sample properties change over time, such as where a trigger system may initiate scanning and analysis of a sample buffer, may likewise be used to partition sample groups and improve detection performance. For example, embodiments where scanning of a buffer is initiated based on changes in sample duration width may be used to break up samples into different groups associated with different parts of a clonic phase of a seizure.

In steps 22 and 24, groups of samples may be qualified using one or more aggregate property values and one or more aggregate qualification thresholds. For example, once one or more groups are organized, as described in step 20, one or more values of one or more aggregate properties of groups may be determined in step 22. Further in step 24, the one or more aggregate property values may be compared to associated aggregate qualification threshold values. For example, in some embodiments, in step 22, property values, including, for example, statistical metrics of a deviation value calculated from duration widths of samples in a group, may be calculated. A deviation value may likewise be calculated from duration widths of parts of samples including, for example, times between elevated portions of samples. A statistical metric of a deviation value calculated from sample duration widths may refer to how far from a single value duration widths of samples are distributed or spread. In some embodiments, one or more metrics associated with how far from a single or average value duration widths of samples are distributed may be determined and used to qualify samples in a group. For example, metrics for how far from a single or average value duration widths of samples in a group are distributed may include, for example, an average deviation, a spread, a percentage deviation, a standard deviation, other suitable metrics, and/or combinations of the aforementioned metrics thereof.

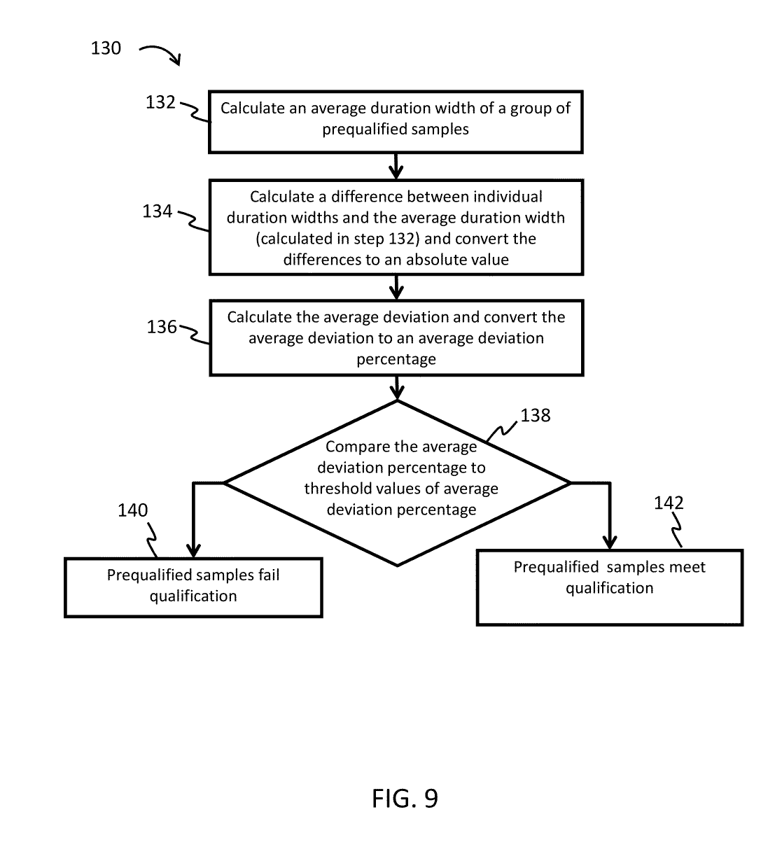

As shown in step 24, calculated property values may be compared to associated aggregate qualification threshold values to qualify the samples in a group. For example, in some embodiments, a standard deviation value, percentage deviation value, or other statistical metric of deviation may be determined from sample duration widths and compared to either or both of a maximum deviation threshold or a minimum deviation threshold. A more detailed description of methods for determining various statistical metrics associated with deviation as may be calculated from duration widths of samples in a group and for applying qualification protocols based thereon is also described in routine 130 of FIG. 9 and in Example 3.

In some embodiments, if a group meets aggregate qualification, all samples within the group may be deemed to meet qualifying conditions selected for the second qualification path of method 10. In some embodiments, including, for example, embodiments configured to search for particular patterns of activity in a collected signal, and embodiments where multiple test groups may be constructed, more than one group of samples may be organized from a set of prequalified samples, including a set of prequalified samples collected within a certain duration of time. In some of those embodiments, one sample may be part of two or more different groups. For example, a sample may be part of one group for which an aggregate property value may be calculated in step 22. In step 24, that aggregate property value may be found to meet an aggregate qualification threshold. The sample may also be part of another group for which an aggregate property value may fail an aggregate qualification threshold. Various protocols may be applied in embodiments herein to rectify any ambiguity in regard to whether a sample as described above is deemed to meet aggregate qualification and whether the sample is included in a second group of qualified-clonic-phase bursts as indicated in step 26. For example, in some embodiments, any sample that is a member of at least one group that meets an aggregate property threshold may be included among a second group of qualified-clonic-phase bursts. Alternatively, where a sample is part of two or more groups including one or more groups that met an aggregate qualification threshold and also one or more groups that failed an aggregate qualification threshold, other procedures may be used to determine if the sample is included in a second group of qualified-clonic-phase bursts. For example, one or more certainty values of group qualification may be determined and applied in deciding if a sample is included in the second group of qualified-clonic-phase bursts.

As shown in step 26, a second group of qualified-clonic-phase bursts may be identified or classified. For example, in some embodiments, samples that are part of at least one group that meets one or more aggregate qualification thresholds in step 24 may be included in the second group of qualified-clonic-phase bursts.

In step 28, a level of qualified-clonic-phase burst activity may be determined. For example, in some embodiments a qualified-clonic-phase burst count or qualified-clonic-phase burst rate may be included in a calculation of activity. In some embodiments, a count or rate may be an integral number. In some embodiments, a count or rate may be certainty weighted. In some embodiments, a certainty weighted value may be compared to a threshold and used to trigger an alarm; however, in some of those embodiments, statistical information associated with or calculated from an integral number of suitably qualified-clonic-phase bursts may be reported to a caregiver such as in the form of a statistics summary. It should be appreciated that whereas a certainty weighted value may be readily treated by a computer processor and used to trigger an alarm, statistics based on an integral number of detected and qualified-clonic-phase bursts may be more intuitively understood and useful to a caregiver. An advantage of some embodiments herein is that individual samples and sample groups may be identified and statistical information for prequalified samples, trains, or qualified-clonic-phase bursts provided to caregivers for analysis. Other methods that process collected signals using some integration algorithms or other transforms may not be configured to measure individual samples as described herein. For example, the identity of individual peaks may be lost because methods may integrate signal over durations wherein the identity of individual peaks and associated samples is lost. And, therefore, those methods may not be configured to provide statistical information based on identified peaks and associated samples as described herein.

In some embodiments, to determine a clonic-phase burst count, samples of an EMG signal that meet qualification conditions for at least one of the first and second qualification paths shown in method 10 may be counted. For example, samples that meet the conditions for qualification in step 14 and step 16 may be identified or marked in step 18 as part of a first group of qualified-clonic-phase bursts. Likewise, samples that are part of a group organized in step 20 and that meet the conditions for qualification in steps 22 and 24 may be identified or marked in step 26 as part of a second group of qualified-clonic-phase bursts. Accordingly, a burst count may include samples that are part of either or both of the aforementioned first and second groups. In some embodiments, to determine a clonic-phase burst count, samples of EMG signal that meet both the first and second qualification paths shown in method 10 may be counted. Therefore, samples included in a burst activity calculation, such as a count, may include either a union of qualified samples of the first and second groups (identified in steps 18 and 26) or an intersection of qualified samples of the first and second groups (identified in steps 18 and 26). Equations for determining a qualified-clonic-phase burst count may be as:

$$\begin{matrix}Qualified count = Set 1 ( \# qualified individually ) ⋂ \\ Set 2 ( \# qualified in aggregate ) \\ = First set of qualified \text{-} clonic \text{-} phase bursts ⋂ \\ Second set of qualified \text{-} clonic \text{-} phase bursts\end{matrix}$$

$$\begin{matrix}Qualified count = Set 1 ( \# qualified individually ) ⋃ \\ Set 2 ( \# qualified in aggregate ) \\ = First set of qualified \text{-} clonic \text{-} phase bursts ⋃ \\ Second set of qualified \text{-} clonic \text{-} phase bursts\end{matrix}$$

In some embodiments, a qualified-clonic-phase burst count may be used to determine if a clonic-phase portion of a seizure is present and to initiate an appropriate response. For example, in some embodiments, an integral count number or rate of qualified-clonic-phase bursts may be compared to a threshold count number or rate. For example, in some embodiments, if a qualified-clonic-phase burst count of between about 2 bursts to about 6 bursts is measured within a time window of about 1 second, a clonic-phase portion of a seizure may be deemed to be likely present. For example, in some embodiments, if the clonic phase of a seizure is detected, an alarm instructing a caregiver that a patient may be having a seizure may be initiated. In some embodiments, a method 10 may be configured to initiate a response if some number of adjacent or nearby windows indicate the presence of qualified-clonic-phase burst activity. For example, in some embodiments, to trigger a response, qualified-clonic-phase burst activity may be required to last for a time duration of about 1 second to about 5 seconds or some other suitable time period. For example, to last for a certain time duration, all time windows or some critical number of time windows within a threshold time duration may be required to show qualified-clonic-phase burst activity.

In some embodiments, a qualified-clonic-phase burst activity level may be determined by collecting an EMG signal and determining a level of qualified-clonic-phase burst activity using the first qualification path of method 10. Some of those embodiments may include use of qualification thresholds selected from a group of qualification thresholds including a minimum duration width threshold and a maximum duration width threshold. In some embodiments, samples may further be qualified if they meet at least one aggregate qualification threshold as may be determined using the second qualification path of method 10. For example, in some embodiments, aggregate qualification threshold values may include a minimum percentage deviation threshold, a maximum percentage deviation threshold, or both. Other thresholds related to how periodic a group of samples is may also be used. Generally, in this disclosure, aggregate properties associated with how periodic a group of samples may be are described in detail. In other applications incorporated herein and commonly owned by Applicant, additional aggregate properties are described. In some embodiments, aggregate properties described in those incorporated references may alternatively or additionally be used to qualify sample groups. For example, as further described in the references incorporated herein, some aggregate properties may include how regular the amplitude of burst data may be or whether burst data matches a level of similarity to one or more spectral waveforms.

In some embodiments, a minimum duration width threshold for an elevated portion of a sample may be between about 25 milliseconds and about 100 milliseconds. For example, a minimum duration width threshold may be about 25 milliseconds, about 50 milliseconds, about 75 milliseconds, or about 100 milliseconds. In some embodiments, a maximum duration width threshold for an elevated portion of a sample may be between about 250 milliseconds and about 400 milliseconds. For example, a maximum duration width threshold may be about 250 milliseconds, about 300 milliseconds, about 350 milliseconds, or about 400 milliseconds.

In some embodiments, an aggregate qualification threshold value may be a percentage deviation of times between elevated portions of samples that are included in a group of samples. For example, in some embodiments, a minimum percentage deviation threshold may be about 5% to about 10%. In some embodiments, a maximum percentage deviation threshold may be about 40% to about 50%.

In some embodiments, a caregiver may be provided a statistical summary of data collected for a patient. For example, statistics for prequalified samples, trains, or fully qualified-clonic-phase bursts may be determined and provided to a caregiver. For example, in some embodiments, trains may include a sample number of between about 3 samples to about 20 samples. In some embodiments, trains may include samples identified over a time period of about 2 seconds to about 5 seconds. In some embodiments, multiple sample trains may be analyzed over time, and changes in sample data between adjacent trains may be calculated. For example, trends in statistics over time may be calculated and displayed using one or more statistics windows. A statistics window may, for example, summarize for a caregiver data over time and may include a number of detected clonic-phase bursts, rate of clonic-phase burst detection, average signal-to-noise ratio (SNR) of detected clonic-phase bursts, spread of SNR of detected clonic-phase bursts, average width for detected clonic-phase bursts, spread of widths for detected clonic-phase bursts, average duration width of periods between elevated portions of detected clonic-phase bursts, spread of duration width of periods between elevated portions of detected clonic-phase bursts, deviation of clonic-phase bursts over any number of trains, frequency characteristics of data, and combinations thereof.

In some embodiments, method 10 may be executed as a “stand-alone” method used for real-time monitoring of a patient for seizure detection. For example, one or more processors configured to execute method 10 may directly or indirectly receive a signal collected from one or more EMG electrodes during patient monitoring. In some embodiments, method 10 may execute as one routine included among one or more other routines in an overall method for real-time monitoring of a patient. In some embodiments, method 10 may be configured for processing EMG data, including, for example, data that was collected at some earlier time. For example, in some embodiments, one or more processors configured to execute method 10 may receive an EMG signal from a storage database or caregiver computer. In some embodiments, steps described in method 10 or in other methods herein may be used in organizing historical or archived patient data, searching the data, and identifying instances, for example, of tonic- or clonic-phase activity.

In some embodiments, method 10 may include a second routine executed together with a first routine. In some of those embodiments, the first routine may be configured to be responsive to tonic-phase activity and/or responsive to motor manifestations that may be present near the start of a seizure. For example, a first routine may be configured to identify seizure-related signals, including, for example, pre-seizure motor manifestations that may indicate that a patient is at elevated risk of progressing to a seizure state. A first routine may, for example, evaluate whether a collected EMG signal exceeds an amplitude or signal-to-noise threshold and/or exceeds such threshold for a specified time period. In some embodiments, the first routine may compare an amplitude value measured in some time interval or time window to a threshold value, and may include a condition that some number of consecutive or nearby time intervals or time windows include an amplitude that exceeds a threshold value in order to determine whether a seizure is present or to determine if the patient exhibits motor activity that may indicate increased risk of progressing to a seizure.

In some embodiments, to improve a signal-to-noise ratio for measurement of collected signals in a first routine, a collected signal may be integrated over a certain interval of time which may, for example, in some embodiments, range for about 0.1 seconds to about 1 second. Selection of longer integration times may configure a routine to be less susceptible to some sources of signal noise thereby improving overall detection sensitivity for weaker signals that may be present near the start of a seizure. However, integration of an EMG signal over significant duration times generally results in a loss of temporal resolution. For example, to reliably compare signal data to a minimum duration width threshold, a routine may be configured to accurately measure a signal that changes at least as fast as the minimum duration width. If such a condition is not met, comparison to the duration width may not produce a meaningful result. For example, a first routine that integrates signals together over an integration window of about 100 milliseconds may have a temporal resolution that is limited to about 200 milliseconds. Therefore, use of such integration windows, which may be beneficial for detecting initial motor manifestations leading to a seizure, may not be appropriate for comparison of signals to duration width thresholds suitable to qualify individual samples as clonic-phase bursts, which may include parts that change more rapidly than reliably measurable when using those integration windows.

Accordingly, in some embodiments, a first routine and a second routine may be configured to have different levels of temporal resolution. For example, a second routine, which may, for example, include steps described in method 10, may be designed to be selective for detection of clonic-phase bursts and may include shorter analysis windows than may be used in some embodiments of first routines described herein. For example, some second routines may include analysis of signal data at the sample rate of sensor collection or collect signals over intervals to facilitate selective detection of discrete samples that may be qualified as clonic-phase bursts. Also by way of example, some but not all first routines described herein may integrate signal within analysis windows of duration of, for example, about 0.1 seconds to about 1 second or some other suitable period, which may be optimized for detection sensitivity and improved signal-to-noise for signals at or before the start of some seizures.

In some embodiments, an amplitude or signal-to-noise threshold setting may be based on a measurement of the maximum signal amplitude an individual may provide during a voluntary muscle contraction. For example, to capture weak motor manifestations that may be present near the start of a seizure or as part of some tonic-phase events, a first routine threshold value of about 2% to about 50% of a maximum voluntary value may be set. In some embodiments, threshold settings may be empirically set which, for some patients, may include threshold settings near the aforementioned maximum voluntary values. In some embodiments, a threshold value may be established based on a baseline signal obtained during one or more resting or calibration periods. For example, a signal amplitude or signal amplitude above a baseline signal may be scaled in units of noise which may, for example, be set based on a standard deviation measured for a baseline signal. A procedure for triggering a response in a first routine may, for example, include scaling the difference between a measured signal amplitude and a baseline signal amplitude in units of standard deviations, and a determination of whether the scaled number of standard deviations exceeds a threshold factor (in units of standard deviations) and/or exceeds that factor for a certain duration of time. A first routine threshold for duration of time may, for example, be about 1 second to about 10 seconds or about 3 seconds to about 7 seconds. A duration time threshold may, for example, extend across some number of time windows. For example, in some embodiments, if an amplitude value exceeds a threshold in all or some critical number of time windows that extend for a period greater than the duration time, a first routine may initiate a response.

Methods that incorporate each of a first routine and a second routine as described herein may be particularly useful in grading and classifying some seizure events. In some embodiments, those methods may be used for detection of seizures in patients particularly difficult to monitor with EMG, including, for example, some patients with high amounts of adipose tissue. Moreover, in some embodiments, those methods may be useful in distinguishing epileptic seizures from other events such as psychogenic or non-epileptic seizure events.

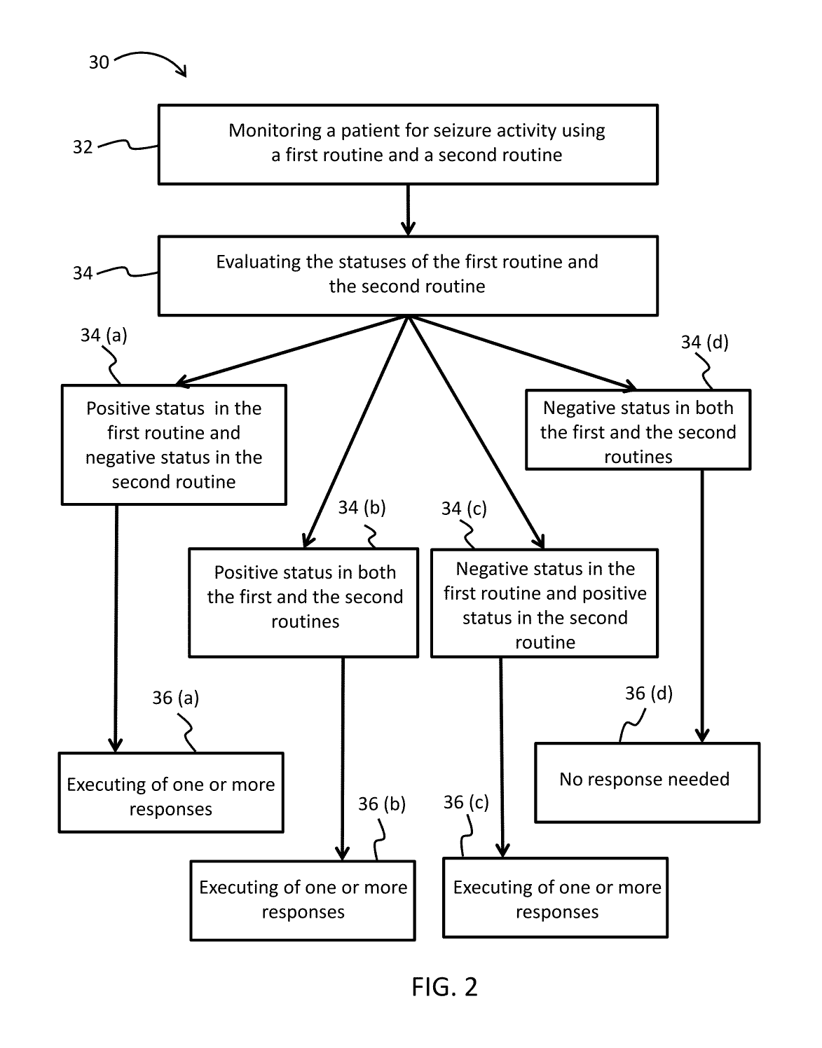

FIG. 2 illustrates embodiments of a method 30 for monitoring a patient for seizure activity. Method 30 may, for example, as shown in step 32, include collection of an EMG signal and analysis of the signal using each of a first routine and a second routine. In some embodiments, the first routine may be configured to be responsive to signals indicating the presence of weak motor manifestations that may be present near the start of a seizure and that may indicate an elevated risk that a patient may progress into a seizure and/or tonic-phase seizure activity. For example, in some embodiments of the first routine, an EMG signal may be collected, and the first routine may analyze the collected signal for events where the collected signal exceeds an amplitude or signal-to-noise threshold and/or exceeds the threshold for a sustained time period. As also shown in step 32, method 30 may further be configured to analyze a collected signal using a second monitoring routine. The second monitoring routine may, for example, include operations described in relation to embodiments of method 10. Accordingly, the second monitoring routine of method 30 may be configured to selectively detect clonic-phase seizure activity. In some embodiments, either or both of the first routine and second routine of method 30 may be executed individually or paired with other routines in an overall method of monitoring a patient for seizure activity.

In a step 34, the statuses of the routines included in method 30 may be evaluated. For example, a managing processor or algorithm may be configured to assess the status of the first and second routines. A managing algorithm may, for example, continuously or at periodic intervals, determine whether either or both of the routines identified activity which the routine was configured to detect. In FIG. 2, statuses of the routines may be described with respect to the list of possible routine outcomes 34(a), 34(b), 34(c), and 34 (d). The outcomes 34(a), 34(b), and 34(c) are combinations of routine statuses in which at least one of the routines shows an affirmative or positive status indicating identification of the activity which the routine is configured to detect. Abnormal patient conditions identified during times in which at least one of the first and second routines show an affirmative status may also be referred to as events. Table 1 further describes classifications for some possible patient conditions and response protocols based on combinations of positive and/or negative routine statuses in first and second routines as they may be configured in some embodiments of method 30.

TABLE 1

| | | | Classification - | |
|---|---|---|---|---|
| Outcome or | Routine 1 - | Routine 2 - | Possible patient | Status/Transmission Protocol |
| Event | Status | Status | state | (Response Protocols) |
| A) | positive | negative | tonic phase or | warning protocol (automatic message only) |
| | | | tonic/non-seizure | |
| B) | negative | positive | clonic phase | emergency protocol (Que alarm message |
| | | | | and send data - enable review of EMG data |
| | | | | by a remote user and/or verification of |
| | | | | event status) |
| C) | positive | positive | clonic phase | emergency protocol (automatic message |
| | | | | along with EMG data to qualified |
| | | | | individual) |
| D) | negative | negative | non-seizure | no transmission |

Of course, if during a certain time period, a managing algorithm finds that neither of the first routine nor the second routine indicates a positive response to the activity it is configured to detect, as shown in outcome 34(d), the patient may not be experiencing a seizure. As shown in 36(d), a significant response may not be needed. Alternatively, during other time periods, as indicated by outcome 34(a), which is associated with response 36(a), the first routine may show a positive status which may, for example, indicate that tonic-phase activity may be present. In some embodiments, first routines described herein may be responsive to tonic-phase activity, but may also respond to clonic-phase activity. However, lack of selectivity for those first routines, when considered individually, may not prevent selective identification of seizure activity and classification of the presence of either or both of the tonic phase and/or clonic phase of a seizure. For example, ambiguity in a patient's status that may arise from a positive first routine outcome may be resolved by considering other routines in method 30. For example, a second routine may be selective for clonic-phase activity. Because a positive status in that routine is not indicated in the outcome 34(a), the combination of routine statuses as described in outcome 34(a) may still, in some embodiments, be used to selectively identify tonic-phase activity.

Some seizures may be brief and/or lack characteristic signatures of more intense seizures such as repetitive motions that may occur in clonic-phase portions of a seizure. At least some of those seizures may be identified in method 30 and with outcome 34(a) or detected using other algorithms or devices, but while such seizures may be detected and may trigger a response, they may, for some patients, present only limited or insignificant risk of injury. Accordingly, in some embodiments, executed response 36(a) may include sending only a warning message to a caregiver or initiate other responses without initiation of an emergency alarm. In contrast, if non-selective seizure detection is made, unnecessary, intrusive, and cost-prohibitive signaling of emergency alarms in response to non-threatening events may be the only appropriate way to respond to such an event detection because the non-threatening event may not be distinguished from other potentially dangerous events. That is, systems that lack selectivity, even if responsive to any of various seizure types, may not be able to customize alarms as described herein. Method 30 may alleviate such concerns for processing data to facilitate a tailored and more cost-effective strategy for patient monitoring.

In some embodiments, systems herein may be configured to execute responses in a manner that may depend upon whether the system is in one of several selectable states. For example, a patient may be given an option to select one or more different monitoring settings based, for example, on whether the patient is in bed sleeping, whether the patient is at home alone, or if the patient is at home with another person. And, depending on whether the patient is in one or another of those states, a monitoring system may select a response protocol that may include either a warning transmission protocol or emergency transmission protocol as further described herein.

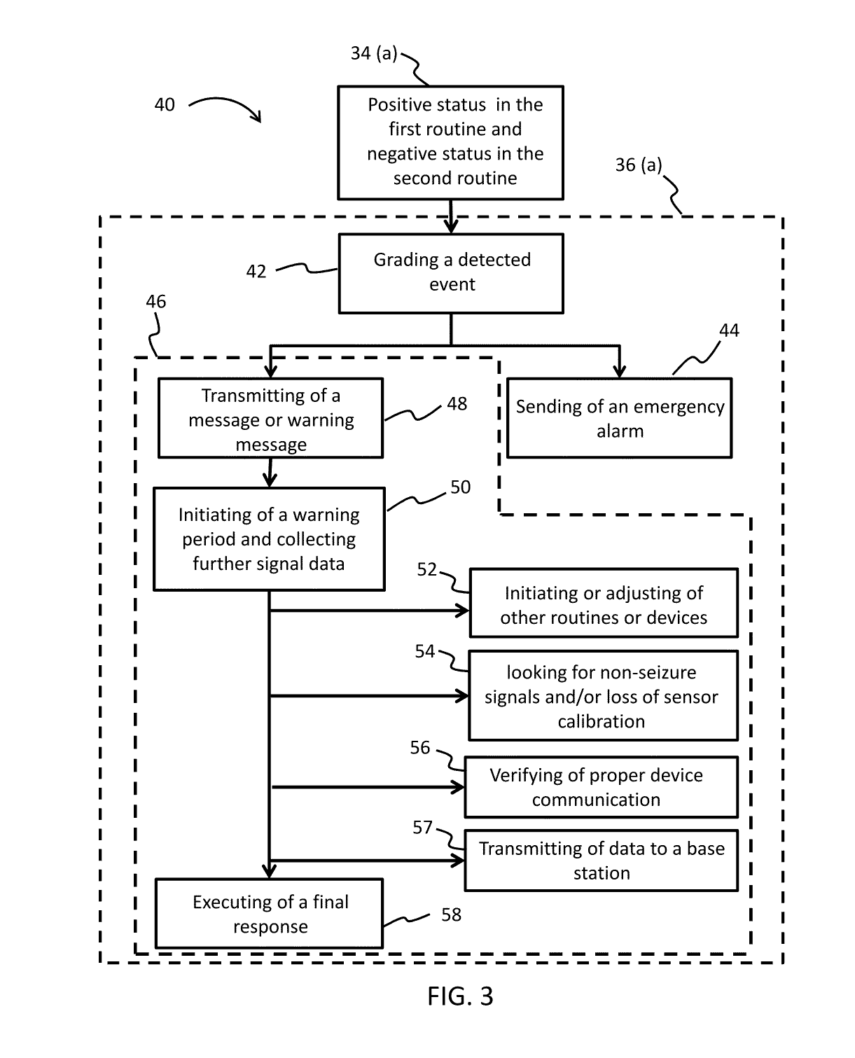

FIG. 3 shows some embodiments of a routine part 40 which may be part of method 30 shown in FIG. 2. Particularly, FIG. 3 describes some embodiments associated with the executing of one or more responses 36(a). In some embodiments, as shown in a step 42, the responses 36(a) may include grading a detected event which may include determining the intensity or severity of the detected event. Grading of the severity of an event may, for example, include normalizing one or more statistical values determined from collected EMG data against other values obtained for other seizure or related events, including, for example, events associated with a particular patient or patient demographic. For example, if a measured amplitude value detected in a first routine is some factor of a previously measured amplitude value, including, for example, an amplitude value measured for a particular patient or patients limited to some demographic group, that factor may be used to grade a detected event. For example, it may be found that a particular event included detection of a signal amplitude that was twice as large as another detected amplitude in another event that was verified to include a tonic-phase seizure, and based, for example, on a group of detected tonic-phase seizures for the patient, the event may be classified with good confidence as a tonic-phase seizure. Other weaker events may be graded differently. For example, an event may include detection of signal amplitude characteristics of motor activity greater than normal background levels of muscle activity, but too low to reliably indicate seizure activity. The event may be associated with motor manifestations that may indicate that the patient is at increased risk of progressing to a seizure. In some embodiments, how an event is graded may be used to classify whether a detected event may have resulted from tonic-phase activity and/or from other motor manifestations, including some motor manifestations which may terminate without progressing to a seizure. That information may, for example, be sent to caregivers and/or otherwise used to initiate a response. For example, based on how an event is graded in step 42, routine part 40 may include a response 44 including initiation of an emergency alarm, or routine part 40 may include one or more other responses (48, 50, 52, 54, 56, 57, and 58) included in a routine part 46.

In some embodiments, one or more amplitude values of a collected EMG signal may be compared to different thresholds in order to grade the severity of a detected signal. For example, a first threshold range of EMG signal amplitude may be from about 5% to about 20% of a value achieved during a maximum voluntary contraction. If that threshold range is achieved, an event may be graded as a motor manifestation that may be indicative of pre-seizure activity, but that does not necessarily indicate that the patient is experiencing a seizure or that a seizure is imminent. Another threshold may be suitable to grade an event as associated with the tonic phase of a seizure. For example, in some embodiments, if an EMG signal amplitude exceeds a threshold level of about 20% or exceeds a level of about 50% of a maximum voluntary contraction, the event may be graded as including a tonic phase of a seizure. In some embodiments, a response may then be based on which threshold is achieved. For example, for some patients, if a higher threshold level is exceeded (or exceeded for some threshold duration of time), an emergency alarm 44 may be triggered. If only a lesser amplitude threshold range is met, routine 40 may trigger other responses included in routine part 46. For some patients, a decision on whether an emergency alarm is issued or if one or more responses associated with response part 46 is executed may depend on whether a system is in one of several selectable states. For example, if the patient is a high-risk or other patient who is at home alone, an emergency alarm 44 may be initiated before or instead of execution of other responses in routine part 46.

In some embodiments or for some graded events, one or more of the different responses included in routine part 46 may be executed. For example, in some embodiments, as shown in a response 48, a message such as a warning message may be transmitted as part of a warning protocol to one or more caregivers. In some embodiments, a time stamp for the event may further be transmitted to a storage database. In this disclosure, an emergency protocol includes any transmission that instructs or is predetermined to instruct that a caregiver physically checks on the health of the patient unless that action is actively canceled by a person. In some embodiments, an emergency protocol may include scheduling transmission of an emergency message, but one or more caregivers may actively terminate the message. For example, a remote caregiver who has reviewed sensor data, reviewed video data of the patient, or has been in contact with the patient, may instruct first responders that an emergency response is not warranted. Some embodiments herein are particularly configured to facilitate such protocols, because, for example, an emergency message may be queued for transmission following a certain period of time, and during that period a caregiver may be provided information suitable to allow them to terminate the transmission if they choose to do so. In contrast to an emergency message, messages that may inform a caregiver that a patient may be experiencing abnormal motor manifestations or that a device may be detecting abnormal activity, but that do not instruct the caregiver to physically check on the health of the patient, may be part of a warning protocol. Of course, one or more caregivers may choose to instruct another caregiver to physically check on a patient in response to a warning message.

In some embodiments, including, for example, following transmission of a message in response 48, one or more of various other responses may be initiated. In some embodiments, a warning period may be initiated as shown in a response 50. During a warning period other responses may be initiated. Those other responses (e.g., 52, 54, 56, and 57) may be configured for selective execution within the warning period or at the start of a warning period. For example, a response 52 may include initiation or adjustment of various routines or devices.

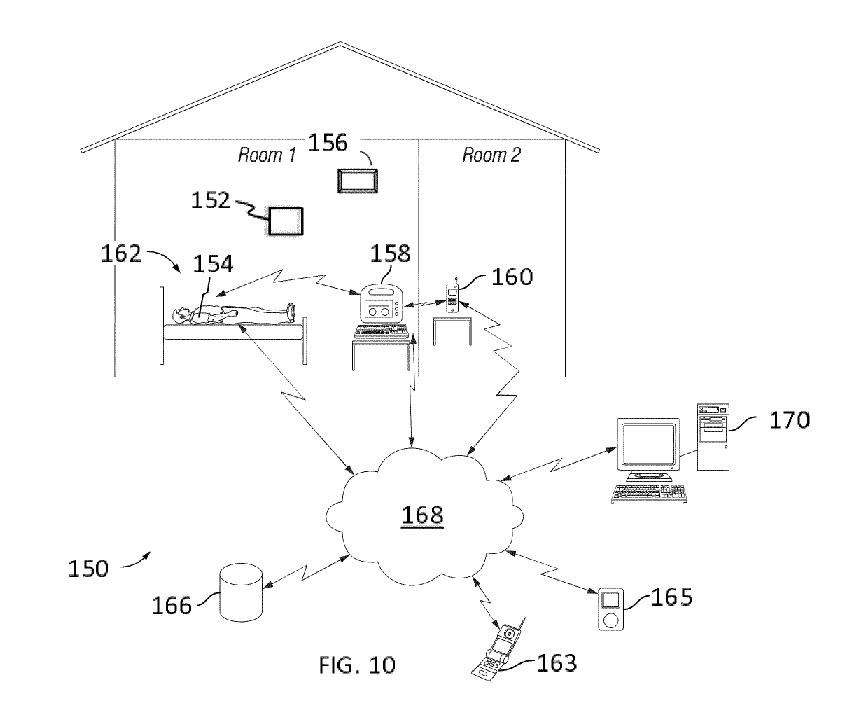

In some embodiments, response 52 may include initiation of one or more routines suitable to detect seizure activity including, for example, clonic-phase seizure activity. In some embodiments, those one or more routines may be additional routines to second routines that may, for example, run continuously in method 30. For example, in some embodiments, second routines executed in method 30 may include detection of clonic-phase activity. Those second routines may include determining whether a group of prequalified samples meets one or more threshold levels of periodicity. Included among advantages of some of those routines is that they may be efficiently executed using a mobile detection device such as detection unit 154 (see FIG. 10) or other mobile device, including those that may have limited processing capability. In some embodiments, other routines may qualify data based on one or more spectral properties of collected data. Some of those other routines may run continuously, but some may beneficially operate in an intermittent manner or as triggered based on initiation of a warning period in step 50. For example, as further described in FIG. 10, some of those routines may include processing of signal data that may be executed by a base station 158, and therefore, may involve transmission of signal data between base station 158 and mobile detection device 154. In some of those routines, transmission of data may include sending volumes of data which, if the routines were executed continuously, may have a negative impact on the battery life of mobile detection device 154. Also, if communication fidelity is low, there may be a risk that transmission of data may delay a response; therefore, again, continuous transmission of significant amounts of data between base station 158 and detection device 154 may not be desired. Accordingly, response 52 may include initiation of routines that for reasons of power consumption and/or other reasons may advantageously be triggered based on the initiation of a warning period as shown in step 50. In some embodiments, routines that may be triggered in step 52 may be highly sensitive to seizure detection, and it may be undesirable to run those routines continuously for reasons other than limiting power consumption. For example, some of those routines, if those routines were continuously executed, may present a greater risk of initiating a false positive detection than desired. In some embodiments, a routine that does run continuously, which may, accordingly, include settings, such as values of threshold settings, which may be configured to minimize risk of inadvertent or false positive detections, may be adjusted in a warning period. For example, threshold settings may be lowered during a warning period to make a routine more sensitive to detection of increased motor activity. Because the warning period may extend for a limited duration, a somewhat higher rate of false positive detections may be accommodated than otherwise would be acceptable if the routine maintained those lowered thresholds indefinitely.

In some embodiments, in step 52, one or more other sensors in addition to sensors configured to detect EMG may be initiated. In some embodiments, in step 52, one or more devices may be activated to treat or terminate a detected seizure. In some embodiments, in step 52, a position of a patient in a monitoring locality may be determined. For example, in some embodiments, the position of a patient may be periodically determined. In step 52, the position of the patient may automatically be updated or a rate at which a patient position is determined may be increased.

A warning period may generally be set to last for some duration. For example, in some embodiments, the warning period may last for about 15 seconds to about 30 seconds or last for another suitable duration. At the completion of a warning period, a final response 58 may be executed. For example, in some embodiments, if elevated signal amplitude is present at the completion of the warning period, the final response may include initiation of an emergency alarm. In contrast, a warning period may terminate without initiation of an emergency alarm. For example, in some embodiments, if only non-seizure motor manifestations are detected during or at completion of the warning period, the warning period may terminate without initiation of an emergency alarm. In some embodiments, a final response may be executed that is an emergency alarm unless it is otherwise identified that the event triggering the warning period was artificial or has ceased. In other embodiments, a final response may be an emergency alarm only if one or more corroborating events indicating possible seizure activity are detected, including, for example, some corroborating events that by themselves would not trigger an emergency alarm. A final response 58 may further include termination or scheduling of termination of other routines or routine adjustments executed at the start of the warning period, including, for example, those described in relation to response 52.

In some embodiments, routine part 46 may include one or more responses 54 wherein a processor is initiated to analyze data for various non-seizure signals. For example, it may be found that a sensor signal is artificially periodic or that the spectral waveform of the signal is not consistent with seizure activity. In addition, in response 54, a routine may be triggered to test whether a sensor may have lost calibration or that sensor performance has otherwise been compromised. For example, in some embodiments, a calibration routine may include testing the contact integrity of one or more electrodes included in the detection device. For example, any of various operations described in Applicant's International Application PCT/US14/61783 may be initiated.

In some embodiments, referring to FIGS. 3 and 10, it may be beneficial to execute a response 54 selectively during a warning period. For example, some of the responses 54 may at least in part be executed by a base station 158 and therefore may include transmission of data between a detection device 154 and the base station 158. Accordingly, as described above, execution of those responses 54 may involve some degree of power consumption. It may be convenient to execute some of the responses 54 at a base station 158 for a number of reasons. For example, generally a base station 158 may include a greater amount of signal memory and/or processing power than detection device 154, and some responses 54, including, for example, some that may look for non-seizure spectral patterns in an EMG signal, may benefit from access to such a base station 158. In addition, some of those routines may apply settings that are adjusted based on the position of a patient within a locale, and in some of those embodiments, environment-specific templates and/or knowledge of the position of the patient may be maintained or calculated by a base station 158. Similarly, some responses 52 that include initiation or adjustment of other routines for seizure detection may also, in some embodiments, involve environment-specific templates. Accordingly, selective execution of those responses 52 and 54 during a warning period may be beneficial.

In some embodiments, the routine part 46 may include execution of one or more responses 56 wherein a system 150 as shown in FIG. 10 may check or establish communication fidelity between a detection unit 154 and a managing device such as a base station 158. Methods for establishing a communication fidelity and/or a risk of a patient moving out of a monitoring locale are described in Applicant's U.S. Provisional Patent Application 62/050,054, the disclose of which is herein fully incorporated by reference. In some embodiments, when communication strength is low or if communication fidelity is at risk of becoming compromised, an emergency alarm may be executed based on an event that otherwise may only include a warning protocol. Such an alarm may, in some embodiments, be initiated before a corroborating seizure event is detected or before the conclusion of a warning period.

In some embodiments, in response 57, EMG data may be sent to a base station 158 during a warning period. For example, in some detection systems, data may be transferred to a base station so that it may then be readily communicated to a caregiver if that information is requested or if that data may be useful to the caregiver. A base station 158 may, for example, be connected to a network via a hard-wired or other high strength connection, and large amounts of data may generally be more efficiently sent from a base station 158 than from a mobile device such as detection unit 154. Therefore, it may be desirable to prepare EMG data for transmission in the event that the data is needed or desired by a caregiver. In some embodiments, EMG data may be sent between a mobile device such as detection unit 154 and base station 158 even before a seizure is verified to be occurring. For example, that data may be sent based on pre-seizure motor manifestations that may or may not progress to a seizure. By queuing data at base station 158, the data may be readily transmitted to a remote caregiver if needed. For example, EMG data stored at base station 158 may be sent to a remote caregiver if a seizure is verified or if requested by a caregiver. In some embodiments, a decision on whether or not to queue data at base station 158 may be based on the communication strength between a mobile detection device such as detection unit 154 and base station 158 and/or may be based on a risk of loss of communication between those devices.

In some embodiments, each of the responses 52, 54, 56, and 57 may execute fully or in part prior to completion of a warning period. Therefore, upon execution of a final response in step 58, the system may generally have access to additional EMG data and/or other information as compared to the information available to the system at the start of a warning period.

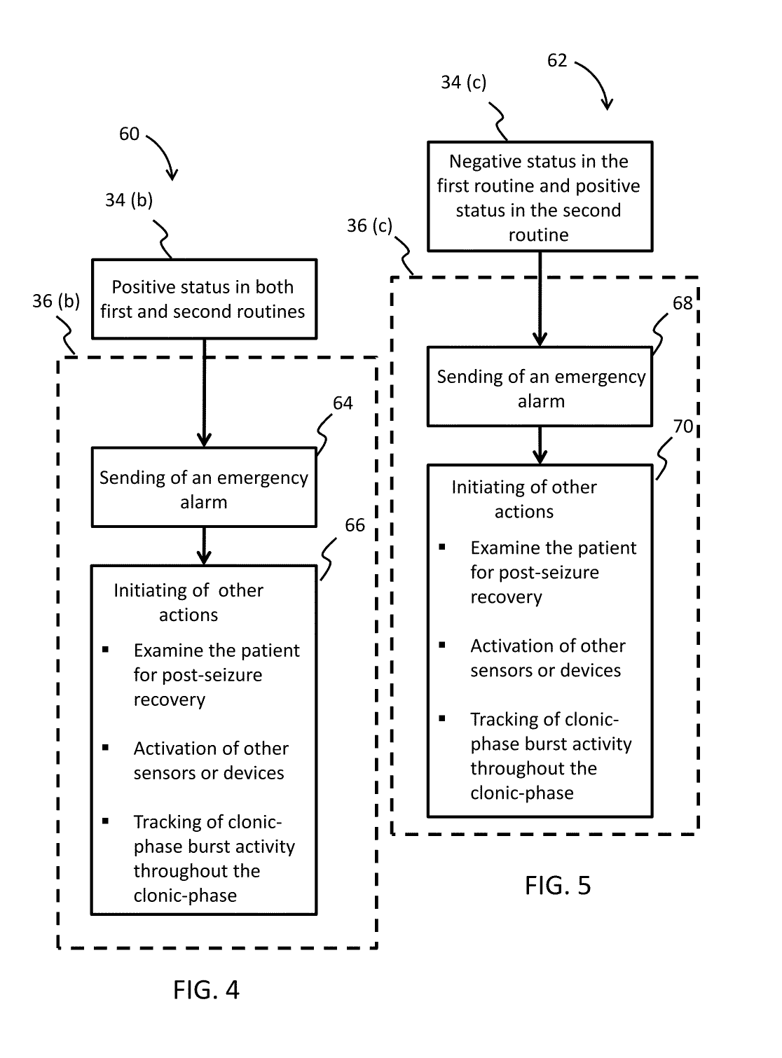

FIG. 4 and FIG. 5 illustrate some embodiments of a routine part 60 and a routine part 62 which may, for example, be part of method 30 shown in FIG. 2. Particularly, FIG. 4 describes routine part 60, which may be associated with one or more responses 36(b) based on the outcome 34(b). FIG. 5 describes the routine part 62, which may be associated with one or more responses 36(c) based on the outcome 34(c). Generally, where clonic-phase activity is detected, an emergency alarm may be initiated as indicated in response 64 (for routine part 60) and response 68 (for routine part 62). Additionally, other responses may be executed. For example, as indicated in response 66 and response 70, one or more routines may be executed to identify signatures of post-seizure recovery. For example, routines as described in Applicant's U.S. application Ser. No. 14/816,924, herein fully incorporated by references may be triggered.

In some embodiments, one or more routines may be used to qualify or prequalify samples of an EMG signal. For example, routines 72, 96, and 118 (described herein in reference to FIGS. 6, 7, and 8) may be used to identify and qualify or prequalify samples of an EMG signal. For example, in some embodiments, the routines 72 and 96 may be used to prequalify samples that may be suitable for further qualification and/or used in a method that counts clonic-phase bursts including, for example, some embodiments of method 10.

FIG. 6 illustrates some embodiments of a routine 72 that may be used individually or in combination with other suitable routines herein to prequalify a sample of an EMG signal. The routine 72 is shown to include a starting or initiation step 74 which, in some embodiments, may include receiving an initiation trigger. The start step 74 may, for example, be initiated based on a detected signal change or a change with respect to background noise. Thus, in some embodiments, routine 72 may run intermittently. In other embodiments, routine 72 may be configured to run continuously without a trigger or activation signal. In step 76, a signal portion may be taken for analysis. The signal portion may include any number of data points collected by an EMG sensor. For example, a sensor may collect data at a rate of about 512/sec, about 1,024/sec, or other rate of data collection. Any number of collected data points may be taken in a signal portion. For example, a data collection rate and data collection number may be selected to achieve a desired temporal resolution for reliable measurement of a time-varying signal. As further described below, routine 72 may operate as a loop. Accordingly, in various steps included in routine 72, either one signal portion or more than one signal portion may be involved. Therefore, in description of the various steps of routine 72, reference may be made to signal portion(s) which may refer to selected or taken data, as shown in step 76, from a single iteration or from multiple iterations or loops of routine 72.

In some embodiments, in step 78, routine 72 may establish whether signal portion(s) taken for analysis include an amplitude that is above a maximum allowed level or threshold setting. For example, it may be desirable, at least in some embodiments, to determine whether the signal amplitude is inappropriately high or achieves a level that is unlikely to correspond with actual seizure activity, but rather may, for example, correspond to an artifact signal such as may be present if a sensor or sensor contact has become unstable or if the detection device is in need of calibration. If the signal amplitude is above the maximum allowed level, as shown in step 80, another routine may be instructed that the signal portion(s) include an amplitude that has exceeded the maximum allowed level. The other routine may, for example, be an algorithm associated with an alarm decision, a routine organized to trigger calibration of a detection unit, or a routine that is looking for signals that may be too regular to be humanly producible. As shown in step 82, routine 72 may then include routing to step 76 for taking a next signal portion or waiting for a next trigger to re-start routine 72.

In a step 84, routine 72 may determine if an amplitude included in the taken signal portion(s) is greater than a reference or background level. For example, it may be determined if an amplitude of the signal portion(s) is greater than background by at least a SNR threshold. The routine 72 may also determine, as shown in step 86, whether the signal portion(s) SNR has exceeded the SNR threshold for the first time within the routine 72. If the signal portion(s) SNR was greater than the SNR threshold for the first time, a timer may be initiated as shown in step 88. A timer may establish a threshold level of duration for qualification or prequalification of samples as may be useful, for example, to prevent transient spikes of activity (e.g., data spikes that may not reflect actual seizures) from being qualified or prequalified. For example, in some embodiments, the timer may run for a period of about 25 milliseconds to about 100 milliseconds or some other suitable value that is shorter than typical of an appropriately qualified-clonic-phase burst. Following the start of a timer in step 88, another signal portion may, for example, be taken as shown in step 76 and another iteration of the routine 72 may then be initiated.

In step 84, if an amplitude included among the signal portion(s) taken for analysis fails to meet the threshold SNR, then a step 90 may be executed. Step 90 may also be executed if the threshold SNR ratio is met in step 84, and where the timer in step 88 was already started (e.g., based on step 86 in a previous iteration). Therefore, step 90 may be executed following execution of either of step 84 or step 86 (depending, for example, on the state of the timer as shown in step 88). In step 90, it may be determined if a time period in which the taken signal portion(s) were above the SNR threshold has exceeded a threshold duration level. If the duration level has been exceeded, taken signal portion(s) may, as shown in step 92, be deemed to be prequalified and the process may exit the routine 72 as shown in a step 94. For example, the taken signal portion(s) may be identified as including a prequalified sample. In some embodiments, prequalified samples identified in routine 72 may be included among prequalified samples included in method 10. In some embodiments, prequalified signal portion(s) exiting routine 72 may be input into routine 96 of FIG. 7. For example, in some embodiments, data processed in both routines 72 and 96 may be included among a group of qualified-clonic-phase bursts. Alternatively, data processed in routines 72 and 96 may be further processed in one or more additional qualification steps. For example, that data may be deemed prequalified and serve as an input in other qualification steps, including, for example, steps described in method 10.

In some embodiments, one or more operations and/or process techniques described herein may be executed in one or more steps of routines 72 and/or 96. For example, in some embodiments, operations, including, for example, signal rectification, filtering, isolation of one or more frequency bands of EMG signal, and/or other suitable processing operations may be executed in one or more steps of routines 72 and/or 96. For example, in some embodiments, an analysis protocol may include a peak detection program, which, for example, after band-pass filtering and rectification, may identify and shape data. Various suitable peak detection techniques may be used (e.g., continuous wavelet transform). For example, in some embodiments, peak detection may include data smoothing techniques (e.g., moving average filter, Savitzky-Golay filter, Gaussian filter, Kaiser Window, various wavelet transforms, and the like), baseline correction processes (e.g., monotone minimum, linear interpolation, loss normalization, moving average of minima, and the like) and application of one or more peak-finding criteria (SNR, detection/intensity threshold, slopes of peaks, local maximum, shape ratio, ridge lines, model-based criterion, peak width, and the like). In some embodiments, isolation of a plurality of frequency bands of EMG signal and calculation of a T-squared value may be executed together with one or more steps of the routines 72 and/or 96.

In some embodiments, some of the aforementioned operations that may be executed in peak detection and/or peak shaping (e.g., smoothing, filtering, peak fitting, and or baseline correction) or other operations described herein may be used to process data following prequalification of the data in either of the routines 72, 96, and 118. For example, following prequalification of one or more samples, a peak detection program may process signal data including the one or more prequalified samples to help shape the signal data and/or identify characteristics of the data that may be used in one or more qualification steps. For example, processing may be used to assist in accurate detection of leading and/or trailing edges of a peak or peak portion of a prequalified sample. Properties of a shaped peak or group of peaks may then be determined. For example, properties associated with duration widths (e.g., of elevated portions of a sample, elevated parts of a sample, and/or adjacent quiet portions of a sample) may be determined from leading and/or trailing peak edges as defined by peak detection. Those duration properties may then, for example, be used in one or more qualification steps, including, for example, as described in routine 10.

By way of example, in some embodiments in which peak data may be identified and shaped following a routine 72, step 84 may include comparing an amplitude of one or more signal portions taken for analysis to a background or noise level that is empirically set or determined from one or more previously collected regions of signal including, for example, regions of signal collected during sensor calibration. An amplitude value may be calculated multiple times in routine 72 (e.g., step 84 may be executed in multiple iterations or loops). An amplitude value may be calculated using all available data (e.g., data from all taken portion(s) of signal as described in step 76) or calculated using only the last signal portion or some preset interval of data among taken signal portions. For example, in some embodiments, irrespective of how many signal portions have been previously taken, an average amplitude value derived from only the last about 10 milliseconds of data or some other suitable amount of data may be used. Some of those embodiments may be used to rapidly prequalify data; following prequalification, that data may further be processed to more accurately determine background data and/or define edges of a peak for improved duration width analysis. For example, any of the aforementioned peak detection operations that may be used to shape data may be executed following prequalification with resulting, suitably shaped, peak data used in one or more qualification steps.

In some embodiments, peak data may be identified and shaped as part of peak detection executed as part of one or more of the routines 72, 96, and 118. In some of those embodiments, step 84 may, for example, include comparing an amplitude value of selected signal portion(s) to a background established using data included among the taken signal portion(s). For example, some number of iterations or loops of routine 72 may be collected before the first time taken signal portion(s) exceed background by at least a SNR, and data from those iterations of routine 72 may be part of a background signal to which elevated portions of signal amplitude may be compared. Elevated portions of signal amplitude may be included among signal portion(s) collected after a SNR threshold is reached. At least an initial estimate of a transition time between background and elevated portions of signal portion(s) may be captured by marking the start of the timer as described in step 88. Therefore, it should be understood that both background signal, which may, for example, be useful for determining characteristics of adjacent portions of quiet signal to a peak or elevated portion, and elevated portions of signal amplitude may be included in signal portion(s) described in routine 72 and also in routine 96. In some embodiments, peak shaping may be executed as part of peak detection executed in step 84 of routine 72 or in step 108 of routine 96. For example, routines 72 and 96 may include use of a peak detection program to smooth a selected or taken signal, perform baseline correction, identify peak edges, identify adjacent baseline regions, and/or perform other processing to fully condition taken signal portion(s) so that properties of the data may be calculated and used to prequalify or qualify signals. Therefore, routines herein, including, for example, routines 72 and 96, may be configured to identify data associated with various parts of prequalified or qualified samples including parts of elevated amplitude and/or adjacent quiet periods of lowered signal amplitude.

In this disclosure, where reference is made to signal amplitude, the signal amplitude may correspond to a collected signal level or absolute value of a collected signal as may be appropriate for a given calculation and/or signal form. Signals collected may, for example, be rectified, and the amplitude of an EMG signal may refer to the magnitude of a signal collected from an EMG sensor and processed by rectification, baseline correction and/or both. In some embodiments, an EMG signal may be processed to isolate one or more frequency bands, and the amplitude of a signal may refer to a magnitude of total signal isolated from the one or more frequency bands. For example, the total power content for various collected bands may be determined.

For many patients, EMG data derived from time periods where the patient is experiencing a seizure may typically exhibit significant amplitude in frequency bands ranging up to about 200 Hz, including, for example, a band from about 60 Hz to about 100 Hz or about 75 Hz to about 85 Hz. In addition, in some embodiments, a series of clonic-phase bursts may be identified by processing data for detection of signal within a band of frequencies between about 1.5 Hz and about 6 Hz. In some embodiments, qualified-clonic-phase bursts may be identified by collecting EMG signals, and spectrally isolating data in one of the aforementioned bands, and where reference is made to the amplitude of a signal, that amplitude may refer to a magnitude of a collected signal isolated from any of the aforementioned bands, other suitable bands, or combinations thereof.

In some embodiments, the magnitude of a statistical value related to levels of motor activity and processed from isolated signal in one or more frequency bands may be determined. For example, in some embodiments, a statistical value may be a T-squared statistical value that may not only be related to levels of motor activity but may also be more sensitive to seizure activity than other values related to motor activity, including, for example, power content determined from one or more bands. Methods of calculating T-squared statistical values are described in detail in Applicant's co-pending U.S. patent application Ser. No. 13/542,596 incorporated herein by reference. And, where reference is made to an amplitude of an EMG signal (or sample of that signal), the magnitude of a statistical metric calculated from an EMG signal such as a T-squared value or other statistical value as described in U.S. patent application Ser. No. 13/542,596 may, in some embodiments, be used. For example, prequalification based on identification of samples including portions of elevated amplitude may include determining a portion of an EMG signal where a T-squared value is elevated. For many patients, a T-squared value may respond more sensitively to seizure activity than may, for example, integrated power values.

FIG. 7 illustrates an embodiment of a routine 96 that may be used individually or in combination with other suitable routines herein to identify or prequalify samples of EMG signal to be qualified. For example, when used in combination with routine 72, routine 96 may facilitate identification or prequalification of samples of an EMG signal that meet each of a minimum duration width criterion and a maximum duration width criterion. Tailoring threshold levels of duration in routines 72 and 96 may, for example, be used to qualify samples of an EMG signal that have signal amplitudes that are elevated over a background level and maintain that elevated signal amplitude for between a minimum duration width of about 50 milliseconds and a maximum duration width of about 300 milliseconds. In some embodiments, qualifying may include use of threshold levels of duration in routines 72 and 96 suitable to select samples that have signal amplitudes that are elevated over a background level and maintain that elevated signal amplitude for between a minimum duration width of about 25 milliseconds and a maximum duration width of about 500 milliseconds.

The routine 96 may include a start or initiation step 98. For example, in some embodiments, initiation step 98 may be executed based on a detected signal change or a change in signal with respect to a background level. Initiation step 98 may alternatively be set to start on a predetermined interval such as may be established by a suitable clock routine. In some embodiments, routine 96 may be initiated once data is input from routine 72. The routine 96 may include taking a signal portion as shown in step 100 which may be a next signal portion added to other signal portions. For example, similarly to routine 72, routine 96 may operate in one or more iterations as a loop. A taken signal portion in step 100 may also be included in other signal portion(s) routed from routine 72. That is, signal portion(s) exiting routine 72 (as shown in step 94) may be input into routine 96, and a taken signal portion in step 100 may be added to other taken signal portion(s) routed from routine 72.

In some embodiments, routine 96 may include step 102 wherein the routine may determine if an amplitude included among taken signal portion(s) exceeds a maximum allowed level. If the maximum level is exceeded, the method may instruct another routine, as shown in step 104, and as shown in step 106, the routine may route back to step 100 for taking a next signal sample or the method may wait for a next trigger before starting over. Alternatively or additionally to execution of step 102, method 96 may include step 108, which may include determining if an amplitude taken among signal portion(s) exceeds background by at least a SNR. If an amplitude included among taken signal portion(s) is greater than background by at least the SNR, method 96 may execute step 110. In step 110, method 96 may determine if the time the signal portion(s) exceed the SNR is greater than a maximum duration level or threshold.

If the maximum duration level or threshold is exceeded, it may be deemed, as shown in step 114, that the signal portion(s) are too long to be indicative of a seizure or clonic-phase portion of a seizure. Appropriate flags may be cleared, and the process may start over by collecting a next signal sample or waiting for an appropriate trigger to re-start the process. If in step 108, it is determined that signal portion(s) do not exceed the background by the SNR threshold level, then taken signal portion(s) may be selected and included among a selected signal sample as shown in step 112 and may exit routine 96 as shown in step 116. Signal samples identified using either or both of routines 72 and 96 may be prequalified samples or qualified samples depending, for example, on how the various routines 72 and 96 or other routines are configured.

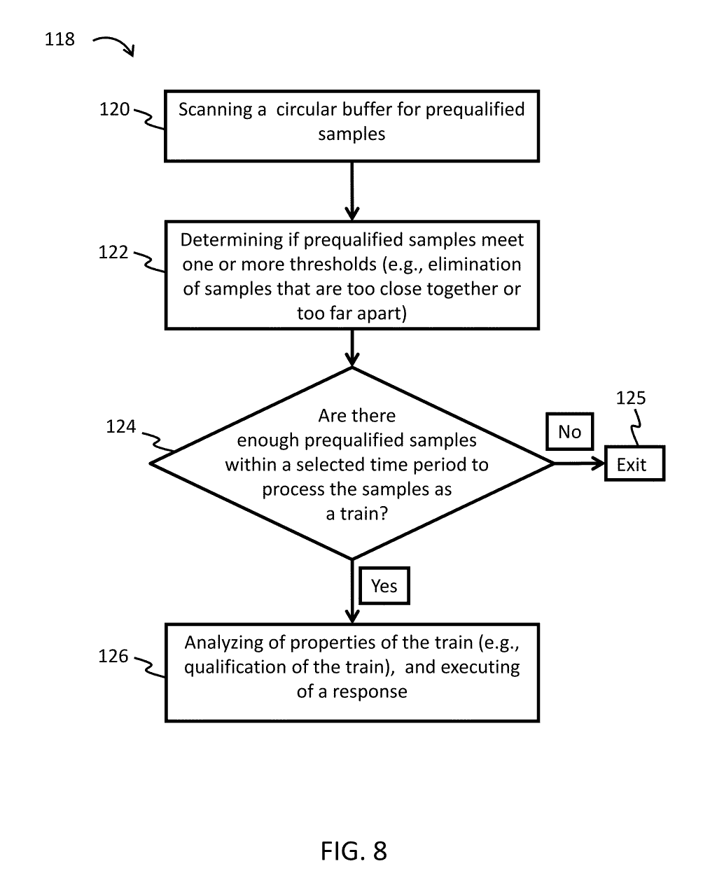

FIG. 8 illustrates an embodiment of routine 118 suitable for organizing or grouping samples of an EMG signal into a train, determining properties of the train, and executing a response if, for example, the train exhibits properties indicating the presence of a seizure. In some embodiments, routine 118 may be executed as part of method 10. For example, in method 10, prequalified samples of an EMG signal may be grouped into a train of adjacent prequalified samples as described in step 20. In some embodiments, those operations may be executed using routine 118 and associated steps 120, 122, and 124. Further in method 10 shown in FIG. 1, one or more aggregate property values of prequalified samples included in the train may be determined as part of qualification as described in steps 24 and 26. In some embodiments, those operations may be executed using routine 118 and associated step 126. In some embodiments, routine 118 may be configured for execution as part of method 10, may operate as an independent method configured for monitoring a patient for seizure activity, or may operate together with other routines in an overall strategy for monitoring of a patient for seizure or seizure-related activity.

As indicated in step 120, routine 118 may scan a circular buffer of data for prequalified signal samples. For example, a circular buffer may store prequalified samples, including, for example, prequalified samples identified using either or both of routines 72 and/or 96. In some embodiments, scanning of the circular buffer may be initiated at predetermined intervals, including, for example, regular intervals based on a clock routine. In some embodiments, scanning may be initiated based on a trigger signal. For example, in some embodiments, each time that data is input into the buffer, which may, for example, occur each time one of routines 72 and/or 96 transfers data to the buffer, scanning may be initiated.

In some embodiments, as indicated in step 122, routine 118 may be configured to determine whether prequalified samples meet one or more threshold conditions. For example, signal samples that are too close together or too far apart may be eliminated from consideration and excluded from a train. Therefore, as also described in method 10, grouping of samples may include a prequalification step which may, for example, be used to prevent stray or aberrant data from being included among a group of samples.

As indicated in step 124, routine 118 may be configured to determine if a suitable number of prequalified samples have been identified to process the prequalified samples as a train. For example, in some embodiments, a number of prequalified samples suitable to be processed as a train may be about 3 samples to about 20 samples. As shown in step 125, if a suitable number of prequalified samples failed to be identified, method 118 may exit. For example, method 118 may stop executing and wait for a next signal or start message suitable to initiate another scan of the buffer. If a suitable number of samples is identified in step 124, as shown in step 126, properties of the train may be determined. In some embodiments, a number of samples suitable to be processed as a train may be selected to encourage grouping of samples in distinct parts of a clonic phase of a seizure. For example, as also described in regard to step 20 of method 10, including a suitable number of samples in a train may be used to create groups of samples where variation of duration widths of periods between samples is associated with natural variation in how physiological events associated with the clonic phase are manifested, but not significantly biased by changes in period length during seizure recovery. Thus, in some embodiments, routine 118 may improve the reliability of aggregate qualification of sample groups. Therefore, in some embodiments, a number of samples deemed suitable for processing as a group or an interval of time deemed suitable for grouping of samples may be selected to encourage reliable aggregate qualification of samples based on group properties.

In step 126, prequalified samples included in one or more trains may be processed to determine properties of the train and to determine a response. For example, one or more aggregate property values of a train may be calculated and compared to associated aggregate property value thresholds. For example, in some embodiments, where routine 118 is executed as part of method 10, qualification of a train may include aggregate qualification of prequalified and grouped samples as described, for example, in steps 22 and 24. As further indicated in step 126, a response may be issued based on analysis of train properties. For example, if an aggregate property value of a train of prequalified samples meets one or more aggregate property thresholds, the prequalified samples may be deemed qualified-clonic-phase bursts. A burst activity level may then be determined. Based on a burst activity level, it may be determined that a patient may be experiencing a seizure and an alarm message may, for example, be issued to a caregiver.

FIG. 9 illustrates embodiments of a routine 130 that may be used to characterize the periodicity of data and evaluate whether data may be indicative of a seizure. In some embodiments, routine 130 may operate as part of method 10. Particularly, routine 130 may be used to execute aggregate qualification of sample groups as described in steps 22 and 24 of method 10.

In step 132 of the exemplary routine 130, an average duration width of a group of prequalified samples or parts of prequalified samples may be calculated. In step 134, individual duration widths may be subtracted from the average duration width, and the absolute values of the differences may be determined. In step 136, the absolute value of the differences may be used in calculation of an average deviation percentage. In this example, the average deviation may be converted to a percentage, although other suitable metrics may also be used to characterize deviation and the variation or distribution of sample duration widths.

In a step 138, the aforementioned average deviation percentage may be compared to threshold values. In some embodiments, threshold values may be taught to the system in operation and may be customized for a particular patient. For example, in a time window (measuring in seconds), nine prequalified samples may be detected. The prequalified samples may be time stamped based, for example, on detection of a leading peak edge, trailing peak edge or other characteristic time point for the samples. For example, a simplified method allows the time around which a sample peak is centered to serve as a time stamp for a sample. The samples may then be referenced to the following exemplary time stamps:

12, 13, 13.75, 14.35, 15, 15.8, 16.2, 16.5, 17.4.

In this example, there would be 8 time periods between the time stamps. Thus, over a time window including the foregoing epoch of 5.4 seconds, there were nine samples with eight duration periods between adjacent time stamps. The average duration period between adjacent time stamps may be calculated as 5.4/8=0.675 seconds per duration period. The individual duration periods may be calculated as follows:

13−12=1

13.75−13=0.75

14.35−13.75=0.6

15−14.35=0.65

15.8−15=0.8

16.2−15.8=0.4

16.5−16.2=0.3

17.4−16.5=0.9

In this example, the individual duration periods may serve as an estimate of a sample's overall duration width. In other embodiments, duration widths of one or more sample parts may be determined. For example, a deviation of time periods of reduced signal adjacent a portion of elevated signal amplitude may be determined. For example, if the sample occurring at 12 seconds included an elevated portion or peak lasting for 0.02 seconds, then the time period between an elevated portion of the sample starting at 12 and the next elevated portion of the sample starting at 13 would be about 0.98 seconds. For example, an adjacent quiet period of a sample may be about 0.98 seconds.

Referring back to the representative example where duration periods between time stamps of adjacent samples are used, a deviation value may be calculated as the difference between an individual duration period and the average duration period. The absolute value of the deviations from the average may be calculated as follows:

|1.0−0.675=0.325

|0.75−0.675|=0.075

|0.6−0.675|=0.075

|0.65−0.675|=0.025

|0.8−0.675|=0.125

|0.4−0.675|=0.275

|0.3−0.675|=0.375

|0.9−0.675|=0.225

Averaging the absolute values may be accomplished as follows:
Sum of all deviations: 0.325+0.075+0.075+0.025+0.125+0.275+0.375+0.225=1.5
Average deviation: 1.5/8=0.1875

The average deviation percentage may be calculated as: 0.1875/0.675=27.8%. It may be deemed that the value is unlikely to be derived from an artificial source. For example, a minimum threshold value of average deviation percentage may be set, for example, to 15%. Accordingly, as shown in step 142, samples included among the group with an average deviation percentage above the minimum threshold value of average deviation percentage may then be deemed to meet aggregate property qualification. For example, referring back to method 10, in some embodiments, samples of the group may then be identified in a second group of qualified-clonic-phase bursts as shown in step 26.

In another simplified example, a train of samples may be time stamped as follows (in seconds):

17, 17.5, 18.02, 18.51, 19.04, 19.56, 20.1, 20.6, 21.13.

So, over a periodicity time window including the foregoing epoch of 4.13 seconds, there were nine samples with eight duration periods between the time stamps for the samples. The average duration period may be calculated as 4.13/8=0.51625 seconds per duration period. The individual duration periods are as follows:

17.5−17=0.5

18.02−17.5=0.52

18.51−18.02=0.49

19.04−18.51=0.53

19.56−19.04=0.52

20.1−19.56=0.45

20.6−20.1=0.5

21.13−20.6=0.53

The absolute value of the deviations from the average may then be determined as follows:

0.5−0.51625|=0.01625

0.52−0.51625|=0.00375

0.49−0.51625|=0.02625

0.53−0.51625|=0.01375

0.52−0.51625|=0.00375

0.45−0.51625|=0.06625

0.5−0.51625|=0.01625

0.53−0.51625|=0.01375

The sum of all deviations may then be calculated as follows:

0.01625+0.00375+0.02625+0.01375+0.00375+0.06625+0.01625+0.01375=1.6

The average deviation is therefore: 1.6/8=0.02. The percentage deviation of this average is thus: 0.02/0.51625=3.87%. This example shows a more regular pattern than in the previous example. If a minimum threshold value of average deviation percentage was set to 15%, then the algorithm would declare that confidence is very low that the train is indicative of a seizure. Accordingly, as indicated in step 140, samples included in the train may fail qualification. Of course, standard deviation calculations or other appropriate metrics related to variability may be substituted for an average deviation percentage. In some embodiments, thresholds may be derived from a particular patient, averaged model values, or some other method. In some embodiments, an average deviation percentage may be compared to a maximum threshold value for average deviation percentage. For example, if an average deviation of a group is less than a maximum threshold value of average deviation, the samples included in the group may be deemed to meet qualification. Or, if an average deviation percentage of a group is greater than a maximum threshold value of average deviation percentage, the samples included in a group may be deemed to fail qualification.

In some embodiments, methods herein may initiate one or more responses, including, for example, initiation of an alarm based on a qualified-clonic-phase burst activity level. In some embodiments, a qualified-clonic-phase burst may be assigned a certainty value which may be used to weight individual qualified-clonic-phase bursts in an algorithm for seizure detection. For example, a qualified-clonic-phase burst may be compared to reference characteristics for a model burst pattern, and based on the similarity of a qualified-clonic-phase burst to the reference characteristics, it may be assigned a greater or lesser weight in a detection algorithm. Certainty values may, in some embodiments, be based on patient or patient demographic values or based on agreement with other qualified-clonic-phase bursts collected during a given time window. In some embodiments, certainty values for qualified-clonic-phase bursts may be based on a combination of metrics including, for example, SNR, width, and amplitude as described in more detail in Applicant's U.S. Pat. No. 8,983,591 incorporated herein by reference.

In some embodiments, a certainty weighted value of qualified-clonic-phase burst activity may be used to detect seizures. However, one or more method routines may also determine a counted number of qualified-clonic-phase bursts and track that number over time or during the course of a seizure. And, in some embodiments, that counted number may be imported into a database for tracking qualified-clonic-phase burst statistics. Importantly, some caregivers may prefer to look at and more readily interpret data based on an integer burst number as opposed to certainty weighted values. That is, counted burst statistics may be more readily interpreted by most caregivers if burst number is a positive integer. In some embodiments, methods herein may not only detect seizures, but also execute routines to look for particular seizure patterns indicative of seizure or seizure-related activity, including those where patterns of peaks most likely to be indicative of seizure activity are isolated from noisy data. And, in some embodiments, by defining patterns with a counted number of qualified bursts, those patterns may be more readily interpreted by a caregiver. Statistics for those identified patterns may also be determined and included in searchable databases accessible to caregivers.

In some embodiments, collected data may be processed in order to create statistical data that may be stored and accessed from a database. Processing collected data as described herein may, for example, facilitate automatic classification and organization of seizure events (e.g., based on type, properties, and/or severity) which may be used in the creation of ordered databases of seizure-related data—an extremely value feature, particularly where video corroboration of events is absent or where individual review of sizeable sets of data by trained professionals, such as medical doctors, would be inconvenient or prohibitively expensive. To that point, it should be understood that one of the major challenges facing the medical community that is presented by methods of monitoring patients in real time is that an enormous amount of data may be generated. Methods that automatically classify events may be used to organize data such that the data may be accessible using data mining techniques. For example, qualified-clonic-phase burst statistics, including, for example, trends in qualified-clonic-phase burst statistics over time, may be particularly useful to a caregiver. Trends in qualified-clonic-phase burst statistics may, for example, be used to differentiate patients who experience epileptic seizures from patients who experience other events, such as non-epileptic psychogenic seizures, which may be commonly confused with seizure events associated with epilepsy.

As described herein, statistics may be generated as data is collected or as it is archived or stored in a database. Furthermore, in some embodiments, a caregiver may create, edit, or apply one or more queries to data already stored in a database. A query may, for example, consider if one or more patterns may be linked to some portion of stored data using methods described herein. A caregiver may actively execute a search query on stored data including, in some embodiments, raw data or compressed data as further described herein. In some embodiments, data may be linked to one or more groups of pre-existing or standard patterns.

In some embodiments, one or more peak detection algorithms may be executed on a data set prior to archiving or storage. And, in some of those embodiments, a compressed version of peak data may be stored together with or alternatively to raw collected data. For example, noise in regions between detected peaks may be depicted as a constant level, proportional to one or more noise metrics. Likewise, data within peak regions may be depicted at a constant level such as the average amplitude present during a peak. Other metrics of detection, such as derivative or inflection data in regions where peak edges were identified, may likewise be stored. Statistical data for peaks, including, by way of nonlimiting example, peak count, peak rate, peak widths, repetition rate, temporal variation, duration of quiet periods, adjacent peaks, and other data, may be stored and made accessible to search queries.

In some embodiments, a database may be configured to enable a user to edit thresholds for a pattern or to create a new pattern. For example, a pattern may include detection of a first burst train defined by various thresholds and a second burst train defined by other thresholds. And, a user may edit or create a pattern to define differences in the aforementioned thresholds. For example, a user may wish to search for all examples of data where burst repetition rate changed by more than 50% or some other number between burst trains over some time period at the start and at the end of a pattern. A user may then select a patient and/or monitoring session and run a query to identify or link stored data to the pattern.

In some embodiments, threshold values used in a method or routine may be set based on one or more training sessions in which a patient may be monitored in a controlled setting. In some embodiments, threshold values may be set based on values found to be useful for all patients or patients of a certain demographic. Method and routine settings may be stored in a template file, which may, in some embodiments, be an adjustable template file. A number of approaches may be used for establishing an initial template file. In some embodiments, a patient may be monitored for a period of time in a hospital or other controlled setting, and data, such as data derived from EMG electrode outputs, may be collected and correlated with the presence or absence of seizures, e.g., general seizure characteristics for an individual may be established. From that data, an operator or software may generate an initial template file or select an appropriate file from a list of pre-generated templates. In some embodiments, an initial template file may be obtained using historical data from a general patient demographic or using historical data for all patients. For example, a patient may be defined by various characteristics including, for example, any combination of age, gender, ethnicity, weight, level of body fat, fat content in the arms, fat content in the legs, or fitness level, or the patient may be defined by other characteristics. The patient's medical history, including, for example, history of having seizures, current medications, or other factors, may also be considered. Once a template file is generated or selected, it may be included in computer memory within a detection unit and base unit, and an individual may use the detection unit in a home-setting.

In some embodiments, a caregiver or patient may provide information related to general seizure characteristics which may be used to adjust method settings. For example, in some embodiments, a patient may receive an alert from the detector unit that a seizure is in progress. An individual, if alert and aware that they are in fact not experiencing a seizure, may be given the option of sending a message to a caregiver and/or to a data storage unit that a false positive was alerted by the system. In some embodiments, an individual may communicate the presence of a false detection by simultaneously pressing two buttons on an attached device, e.g., the detection unit or another unit. Of course, the requirement that an individual simultaneously press two buttons may minimize the risk that an inadvertent signal is sent. Any other suitable approach to minimize inadvertent messages may also be used. A message sent in this manner, e.g., sent to a storage facility from a patient, may include a time stamp to correlate a false positive event with the data which initiated the false positive event. Such information may be stored in a data storage facility and/or otherwise used to adjust method or routine settings.

In some embodiments, for example, a log of false positive events in a first routine may be logged together with a log of actual seizure events. In some embodiments, that information may be used to adjust a sensitivity of a first routine. For example, the sensitivity may be adjusted from initial settings so that a desired ratio of first routine responses may be associated with non-seizure and seizure activity.

A variety of suitable systems may be used for collecting large amounts of EMG and other patient-related data, organizing such data for system optimization, and initiating an alarm or other response based on suspected seizure activity. FIG. 10 illustrates an exemplary embodiment of such a system that may be configured to monitor a patient for seizure activity using the methods described herein. In the embodiment of FIG. 10, a seizure detection system 150 may include a video camera 152, a detection unit 154, an acoustic sensor 156, a base station 158, and an alert transceiver 160. The detection unit 154 may comprise one or more EMG electrodes capable of detecting electrical signals from muscles at or near the skin surface of a patient 162 and delivering those electrical EMG signals to a processor for processing. The EMG electrodes may be attached to the patient 162, and may, in some embodiments, be implanted within the tissue of the patient 162 near a muscle that may be activated during a seizure. Implanted devices may, for example, be particularly amenable for some patients where EMG signals may typically be weak, such as patients with significant adipose tissue. The base station 158 may comprise a computer capable of receiving and processing EMG signals from the detection unit 154 and/or acoustic data from the acoustic sensor 156, determining from the processed EMG and/or acoustic signals whether a seizure may have occurred, and sending an alert to a caregiver. The alert transceiver 160 may be carried by, or placed near, a caregiver to receive and relay alerts transmitted by the base station 158 or transmitted directly from the detection unit 154. Other components that may be included in the system 150, including for example, wireless communication devices 163 and 165, storage database 166, electronic devices for detecting changes in the integrity of an electrode skin interface, and one or more environmental transceivers are also described in U.S. Pat. No. 8,983,591 and other references incorporated herein.

In using the apparatus of FIG. 10, the patient 162 susceptible to epileptic seizures may, for example, be resting in bed, or may be at some other location as daily living may include, and may have a detection unit 154 in physical contact with or in proximity to his or her body. The detection unit 154 may be a wireless device so that the patient 162 may be able to get up and walk around without having to be tethered to an immobile power source or to a bulkier base station 158. For example, detection unit 154 may be woven into a shirt sleeve, may be mounted to an armband or bracelet, or may be an implanted device. In other embodiments, one or more detection units 154 may be placed or built into a bed, a chair, an infant car seat, or other suitable clothing, furniture, equipment or accessories used by persons susceptible to seizures. The detection unit 154 may comprise a simple sensor, such as an electrode, that may send signals to base station 158 for processing and analysis, or may comprise a “smart” sensor having some data processing and storage capability. In some embodiments, a simple sensor may be connected via wire or wirelessly to a battery-operated transceiver mounted on a belt or other garment or accessory worn by the patient 162.

The system 150 may monitor the patient 162, for example, while resting, such as during the evening and nighttime hours or during the daytime. If the detection unit 154 on the patient 162 detects a seizure, the detection unit 154 may communicate wired or wirelessly, e.g., via a communications network or wireless link, with the base station 158 to a remote cell phone or desktop device via Bluetooth or other signal or simultaneously to a base station 158 and remote cell phone or other device. In some embodiments, a detection unit 154 may send some signals to the base station 158 for further analysis. For example, the detection unit 154 may process and use EMG signals (and optionally or additionally, or in some embodiments ECG, temperature, orientation sensors, saturated oxygen, and/or audio sensor signals) to make an initial assessment regarding the likelihood of occurrence of a seizure, and may send those signals and its assessment to base station 158 for separate processing and confirmation. If the base station 158 confirms that a seizure is likely occurring, then base station 158 may initiate an alarm for transmission over a network 168 to alert a caregiver by way of email, text, phone call, or any suitable wired or wireless messaging indicator. It should be appreciated that detection unit 154 may, in some embodiments, be smaller and more compact than base station 158 and it may be convenient to use a power supply with only limited strength. Therefore, it may be advantageous, in some embodiments, to control the amount of data that is transferred between detection unit 154 and base station 158 as this may increase the lifetime of any power supply elements integrated in or associated with the detection unit 154. In some embodiments, if one or more of detection unit 154, base station 158, or a caregiver, e.g., a remotely located caregiver monitoring signals provided from base station 158, determines that a seizure may be occurring, a video camera 152 may be triggered to collect video information of the patient 162.

In some embodiments, a single sensor may be used to monitor a patient for EMG activity. In other embodiments, at least two sensors may be attached to a patient. In some embodiments, sensors may be configured such that a patient when sleeping has at least one sensor that is not disposed between a surface of the bed and the patient's body. For example, a patient may have sensors on opposite arms such that if the patient sleeps on either the left or right side of his or her body at least one sensor may typically not be disposed against the bed. A monitoring system may, for example, be configured to initiate a response if either or both of muscles on the patient's left or right side are suitably activated to show seizure activity, and in some embodiments, a detected event may be classified based on symmetry of signals or lack of symmetry between the left and right sides of a patient's body.

The base station 158, which may be powered by a typical household power supply and may contain a battery for backup, may have more processing, transmission and analysis power available for its operation than detection unit 154 and may be able to store a greater quantity of signal history and evaluate a received signal against that greater amount of data. The base station 158 may communicate with an alert transceiver 160 located remotely from base station 158, such as in the bedroom of a family member, or to a wireless device 163, 165 carried by a caregiver or located at a work office or clinic. The base station 158 and/or transceiver 160 may send alerts or messages to caregivers or medical personnel via any suitable means, such as through network 168 to a cell phone 163, PDA 165 or other client device. The system 150 may thus provide an accurate log of seizures, which may allow a patient's physician to understand more quickly the success or failure of a treatment regimen. Of course, base station 158 may simply comprise a computer having installed a program capable of receiving, processing, and analyzing signals as described herein and capable of transmitting an alert. In other embodiments system 150 may simply comprise, for example, EMG electrodes and a smartphone, such as an iPhone, configured to receive EMG signals from the electrodes for processing the EMG signals as described herein using a program application installed on the smart phone. In further embodiments, so-called “cloud” computing and storage may be used via network 168 for storing and processing the EMG signals and related data. In yet other embodiments, one or more EMG electrodes may be packaged together as a single unit with a processor capable of processing EMG signals as disclosed herein and sending an alert over a network. In other words, the apparatus may comprise a single item of manufacture that may be placed on a patient and that does not require a base station 158 or separate transceiver 160, or the base station 158 may be a smartphone or tablet, for example.

In the embodiment of FIG. 10, the EMG signal data may be sent to a remote database 166 for storage. In some embodiments, EMG signal data may be sent from a plurality of epileptic patients to a remote database 166 or central database and “anonymized” to provide a basis for establishing and refining generalized “baseline” sensitivity levels and signal characteristics of an epileptic seizure. The database 166 and base station 158 may be remotely accessed via network 168 by a remote computer 170 to allow for updating of detector unit 154 and/or base station 158 software and to allow for data transmission. The base station 158 may generate an audible alarm, as may a remote transceiver 160. All wireless links may be two-way for software and data transmission and message delivery confirmation. The base station 158 may also employ one or more of the messaging methods listed above for seizure notification. The base station 158 or detection device 154 may provide an “alert cancel” button to terminate an incident warning.

In some embodiments, a transceiver may additionally be mounted within a unit of furniture or some other structure, e.g., an environmental unit or object. If a detection unit 154 is sufficiently close to that transceiver, such a transceiver may be capable of sending data to a base station 158. Thus, base station 158 may be aware that information is being received from that transceiver, and therefore base station 158 may identify the associated environmental unit. In some embodiments, a base station 158 may select a specific template file, e.g., such as including threshold values and other data as described further herein, that is dependent upon whether or not it is receiving a signal from a certain transceiver. Thus, for example, if base station 158 receives information from a detector and from a transceiver that is associated with a bed or crib, it may treat the data differently than if the data is received from a transceiver associated with another environmental unit, such as, for example, clothing typically worn while an individual may be exercising, or an item close to a user's sink, where for example a patient may brush his or her teeth. More generally, a monitoring system may, in some embodiments, be configured with one or more elements with global positioning system (GPS) capability, and GPS position information may be used to adjust one or more routines that may be used in a detection algorithm.

The embodiment of FIG. 10 may be configured to be minimally intrusive to use while sleeping or minimally interfere in daily activities, may require a minimum of electrodes such as one or two, may require no electrodes to the head, may detect a seizure with motor manifestations, may alert one or more local and/or remote sites of the presence of a seizure, and may be inexpensive enough for home use.

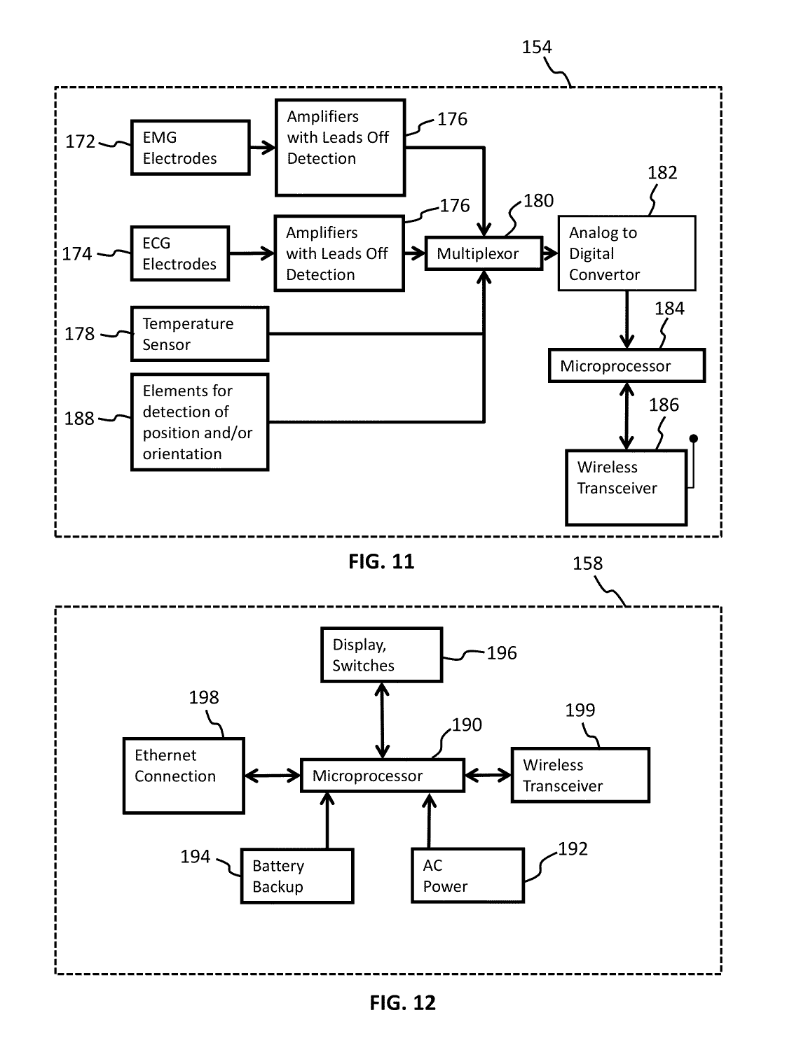

FIG. 11 illustrates an embodiment of a detection unit 154 or detector. The detection unit 154 may include EMG electrodes 172 and may also include ECG electrodes 174. The detection unit 154 may further include amplifiers with leads-off detectors 176. In some embodiments, one or more leads-off detectors may provide signals that indicate whether the electrodes are in physical contact with the person's body or otherwise too far from the person's body to detect muscle activity, temperature, brain activity or other patient phenomena. In some embodiments, the detection unit 154 may further include one or more elements 188, such as solid state MEMS structures, configured for detection of position and/or orientation of the detection unit 154. For example, an element 188 may include one or more micromachined inertial sensors such as one or more gyroscopes, accelerometers, magnetometers or combinations thereof.

The detection unit 154 may further include a temperature sensor 178 to sense the person's temperature. Other sensors (not shown) may be included in the detection unit, as well, such as accelerometers and microphones. Signals from electrodes 172 and 174, temperature sensor 178 and other sensors may be provided to a multiplexor 180. The multiplexor 180 may be part of the detection unit 154 or may be part of the base station 158 if the detection unit 154 is not a smart sensor. The signals may then be communicated from the multiplexor 180 to one or more analog-to-digital (A-D) converters 182. The analog-to-digital converters may be part of detection unit 154 or may be part of base station 158. The signals may then be communicated to one or more microprocessors 184 for processing and analysis as disclosed herein. The microprocessor 184 may be part of detection unit 154 or may be part of base station 158. The detection unit 154 and/or base station 158 may further include memory of suitable capacity. The microprocessor(s) 184 may communicate signal data and other information using a transceiver 186. Communication by and among the components of detection unit 154 and/or base station 158 may be via wired or wireless communication.

Of course, the exemplary detection unit of FIG. 11 may be differently configured. Many of the components of the detector of FIG. 11 may be in base station 158 rather than in detection unit 154. For example, detection unit 154 may simply comprise an EMG electrode 172 in wireless communication with a base station 158. In such an embodiment, A-D conversion and signal processing may occur at base station 158. If an ECG electrode 174 is included, then multiplexing may also occur at base station 158.

In another example, detection unit 154 of FIG. 11 may comprise an electrode portion having one or more of the EMG electrode 172, ECG electrode 174, element 188, and temperature sensor 176 in wired or wireless communication with a small belt-worn transceiver portion. The transceiver portion may include a multiplexor 180, an A-D converter 182, microprocessor 184, transceiver 186 and other components, such as memory and I/O devices (e.g., alarm cancel buttons and visual display).

FIG. 12 illustrates an embodiment of a base station 158 that may include one or more microprocessors 190, a power source 192, a backup power source 194, one or more I/O devices 196, and various communications means, such as an Ethernet connection 198 and wireless transceiver 199. The base station 154 may have more processing and storage capability than detection unit 154 and may include a larger electronic display for displaying EMG signal graphs for a caregiver to review EMG signals in real-time as they are received from the detection unit 154 or historical EMG signals from memory. The base station 158 may process EMG signals and other data received from detection unit 158. If the base station 158 determines that a seizure is likely occurring, it may send an alert to a caregiver via transceiver 199.

Various devices in the apparatus of FIGS. 10-12 may communicate with each other via wired or wireless communication. The system 150 may comprise a client-server or other architecture, and may allow communication via network 168. Of course, system 150 may comprise more than one server and/or client. In other embodiments, system 150 may comprise other types of network architecture, such as a peer-to-peer architecture, or any combination or hybrid thereof.

Generally, the devices of a seizure detection system may be of any suitable type and configuration to accomplish one or more of the methods and goals disclosed herein. For example, a server may comprise one or more computers or programs that respond to commands or requests from one or more other computers or programs, or clients. The client devices may comprise one or more computers or programs that issue commands or requests for service provided by one or more other computers or programs, or servers. The various devices in FIG. 10, e.g., 152, 154, 156, 158, 163, 165, 166, and/or 170, may be servers or clients depending on their function and configuration. Servers and/or clients may variously be or reside on, for example, mainframe computers, desktop computers, PDAs, smartphones (such as Apple's iPhone™ Motorola's Atrix™ 4G, Motorola's Droid™, Samsung's Galaxy S™, Samsung's Galaxy Note™, and Research In Motion's Blackberry™ devices), tablets (such as Sony's Xperia™ Samsung's Galaxy Tab™, and Amazon Kindle™) netbooks, portable computers, portable media players with network communication capabilities (such as Microsoft's Zune HD™ and Apple's iPod Touch™ devices), cameras with network communication capabilities, smartwatches, wearable computers, and the like.

A computer may be any device capable of accepting input, processing the input according to a program, and producing output. A computer may comprise, for example, a processor, memory and network connection capability. Computers may be of a variety of classes, such as supercomputers, mainframes, workstations, microcomputers, PDAs and smartphones, according to the computer's size, speed, cost and abilities. Computers may be stationary or portable and may be programmed for a variety of functions, such as cellular telephony, media recordation and playback, data transfer, web browsing, data processing, data query, process automation, video conferencing, artificial intelligence, and much more.

A program may comprise any sequence of instructions, such as an algorithm, whether in a form that can be executed by a computer (object code), in a form that can be read by humans (source code), or otherwise. A program may comprise or call one or more data structures and variables. A program may be embodied in hardware or software or a combination thereof. A program may be created using any suitable programming language, such as C, C++, Java, Perl, PHP, Ruby, SQL, and others. Computer software may comprise one or more programs and related data. Examples of computer software include system software (such as operating system software, device drivers and utilities), middleware (such as web servers, data access software and enterprise messaging software), application software (such as databases, video processors and media players), firmware (such as device specific software installed on calculators, keyboards and mobile phones), and programming tools (such as debuggers, compilers and text editors).

Memory may comprise any computer-readable medium in which information can be temporarily or permanently stored and retrieved. Examples of memory include various types of RAM and ROM, such as SRAM, DRAM, Z-RAM, flash, optical disks, magnetic tape, punch cards, and EEPROM. Memory may be virtualized and may be provided in or across one or more devices and/or geographic locations, such as RAID technology. An I/O device may comprise any hardware that can be used to provide information to and/or receive information from a computer. Exemplary I/O devices include disk drives, keyboards, video display screens, mouse pointers, printers, card readers, scanners (such as barcode, fingerprint, iris, QR code, and other types of scanners), RFID devices, tape drives, touch screens, cameras, movement sensors, network cards, storage devices, microphones, audio speakers, styli and transducers, and associated interfaces and drivers.

A network may comprise a cellular network, the Internet, intranet, local area network (LAN), wide area network (WAN), Metropolitan Area Network (MAN), other types of area networks, cable television network, satellite network, telephone network, public networks, private networks, wired or wireless networks, virtual, switched, routed, fully connected, and any combination and subnetwork thereof. The network may use a variety of network devices, such as routers, bridges, switches, hubs, repeaters, converters, receivers, proxies, firewalls, translators and the like. Network connections may be wired or wireless and may use multiplexers, network interface cards, modems, IDSN terminal adapters, line drivers, and the like. The network may comprise any suitable topology, such as point-to-point, bus, star, tree, mesh, ring, and any combination or hybrid thereof.

Wireless technology may take many forms such as person-to-person wireless, person-to-stationary receiving device, person-to-a-remote alerting device using one or more of the available wireless technologies such as ISM band devices, WiFi, Bluetooth, cell phone SMS, cellular (CDMA2000, WCDMA, etc.), WiMAX, WLAN, and the like.

Communication in and among computers, I/O devices, and network devices may be accomplished using a variety of protocols. Protocols may include, for example, signaling, error detection and correction, data formatting, and address mapping. For example, protocols may be provided according to the seven-layer Open Systems Interconnection model (OSI model) or the TCP/IP model.

Additional information related to the methods and apparatus described herein may be understood in connection with the examples provided below. In Example 1, EMG data is shown for a patient monitored for seizure activity using EMG electrodes and showing a detected seizure. Some representative embodiments of methods suitable to detect the seizure in Example 1 are described in the Examples 2-4. Example 4 further shows group data for a number of detected seizures for various patients and describes changes in EMG data throughout the course of the clonic phase of a number of detected seizures.

### Example 1

In this Example 1, a patient susceptible to seizures was monitored for seizure activity using EMG electrodes. A sensor was placed on the patient's biceps, EMG signals collected, the collected signals were analyzed for the presence of seizure activity, and a seizure was detected. The collected EMG signals may, for example, be analyzed using a combination of routines including a first routine sensitive to initial motor manifestations present near the start of a seizure and a second routine selective for detection of activity related to the clonic phase of a seizure.

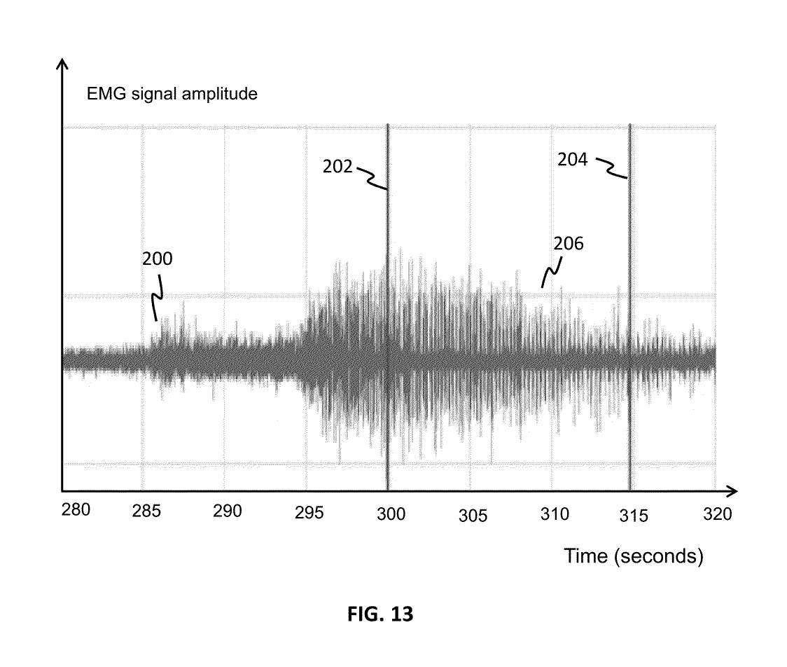

EMG data for the seizure detected in this Example 1 is shown in FIG. 13. At a time marker 200, located at about 285 seconds, an increase in EMG signal amplitude is evident. That increase in signal amplitude may be identified in a first routine which may be configured to detect weak motor manifestations that may be present near the start of a seizure. For this patient, those initial signatures are relatively weak, and based on the amplitude of EMG signals it may be difficult to differentiate motor manifestations that may precede a seizure from other motor manifestations, such as non-seizure related movements and/or baseline signals. Selectivity for detection of seizure events over other events may be somewhat improved by adding the condition that the EMG signal maintain an elevated amplitude for a sustained period of time or by integrating the EMG signal over analysis windows of extended duration. For example, analysis windows of about 100 milliseconds or longer may be used, and signals collected in an analysis window may be integrated to determine an integrated amplitude value. To initiate a response, a routine may then, for example, determine if amplitude values in some number of consecutive windows or a number of analysis windows over a time period meets a threshold value.

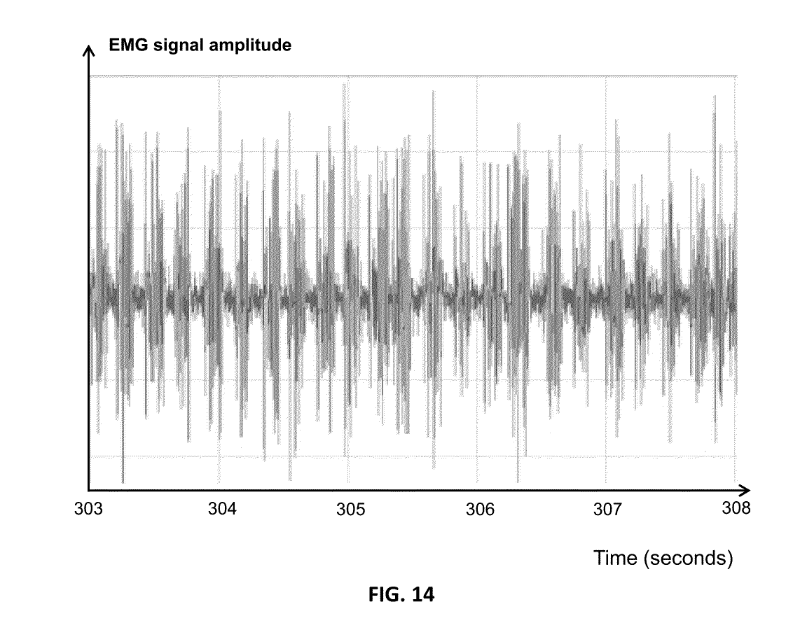

FIG. 13 also shows a first solid marker 202 at a time of about 300 seconds and a second solid marker 204 at a time of about 315 seconds. The markers 202 and 204 respectively identify times at about the start of the clonic phase (marker 202) and near the end of the clonic phase (marker 204) of the detected seizure. For this patient, video monitoring was used to confirm the presence of clonic-phase activity. As also shown in FIG. 13, transient elevations of EMG signal are present, i.e., the EMG data shows a characteristic pattern of transient elevations during the clonic phase. That pattern is also shown in more detail in FIG. 14, which shows the amplitude of collected signals over a time period of about 5 seconds (between about 303 seconds to about 308 seconds), and which clearly displays a series of signal elevations. Those signal elevations or peaks may, as described below, be detected and used to qualify clonic-phase bursts. A qualified-clonic-phase burst count or rate may then be used to initiate a response to detection of clonic-phase activity.

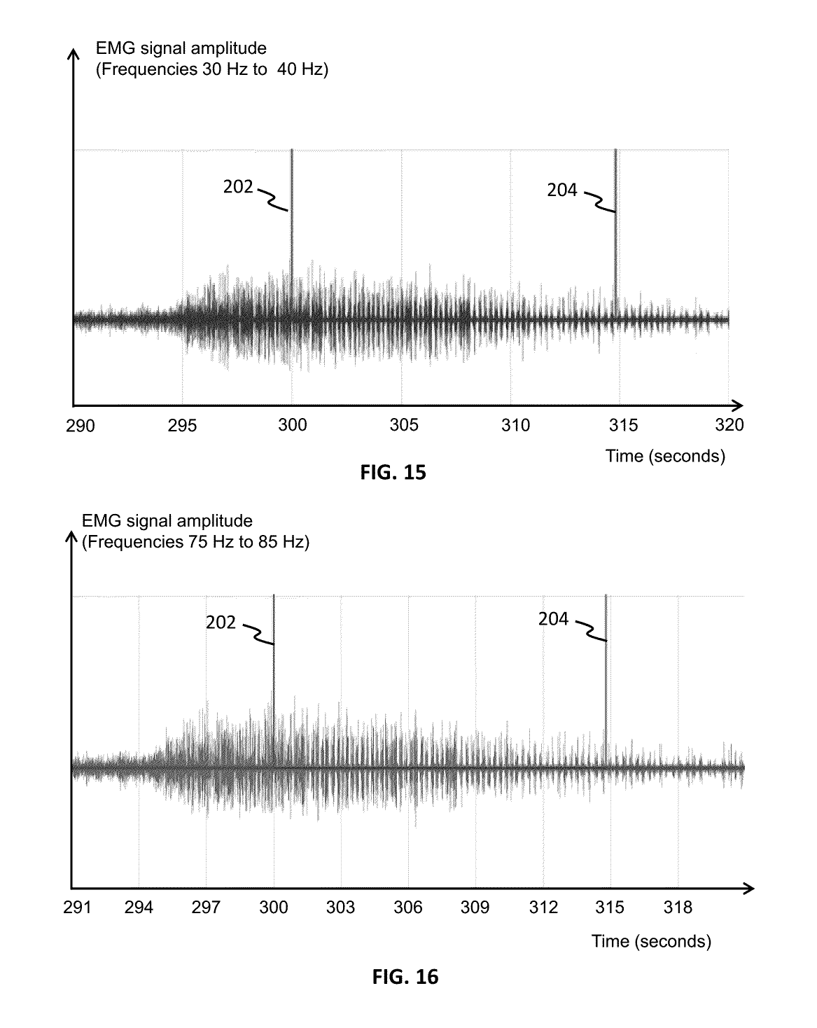

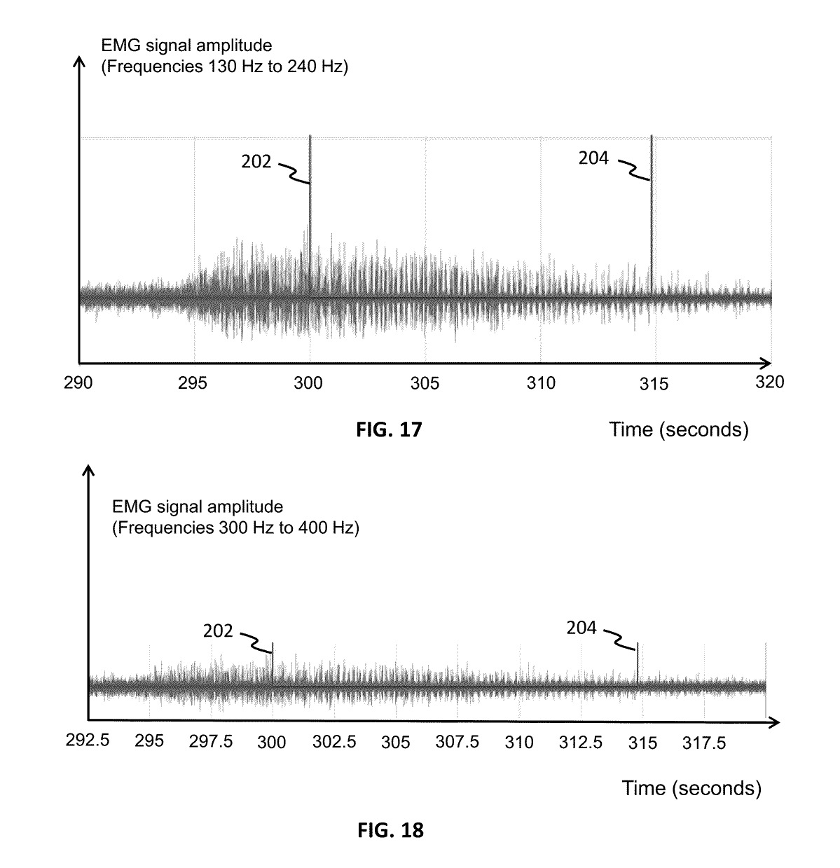

As found for most patients, for this patient, EMG data collected during a seizure episode shows significant amplitude within a number of frequency bands, including, for example, a band from about 75 to about 85 Hz. Selection of one or more frequency bands for use in a monitoring routine may be made to encourage differentiation of seizure events from some non-seizure events. For the patient in Example 1, amplitudes of EMG signal collected in a number of frequency bands were isolated and tracked over time. In FIGS. 15-18, amplitude data isolated from frequency bands from about 30 to 40 Hz (FIG. 15), 75 Hz to 85 Hz (FIG. 16), 130 Hz to 240 Hz (FIG. 17), and 300 Hz to 400 Hz (FIG. 18) are shown. The amplitude of EMG signals over any combination of the bands herein may, for example, be used to evaluate the patient for the presence of seizure activity. That is, EMG amplitude (or intensity) may be evaluated using signals in any of the aforementioned bands or combinations thereof. However, for some other patients, other bands may be selected.

### Example 2

In this Example 2, an embodiment of a method of analyzing collected EMG signals is described. The method may, for example, be used to detect the seizure activity described above for the patient in Example 1. In Example 2, a first routine may be executed to analyze EMG data for the presence of sustained EMG activity, the presence of which may indicate initial motor manifestations of a seizure or that a patient may be at elevated risk of having a seizure. That routine may include one or more threshold settings, and if the thresholds are met, a warning response may be initiated. A second routine may also be executed. The second routine may be configured to be selective for detection of clonic-phase seizure activity. For example, in Example 2, the second routine may determine the presence of a qualified-clonic-phase burst count, which may be compared to a threshold count and used to determine if clonic-phase seizure activity is present.

Table 2 shows, by way of example, some settings that may be applied to monitor a patient using the first routine of Example 2.

TABLE 2

| Routine Setting | Value |
|---|---|
| Frequency band selected for routine 1 | Full Range/ |
| | 75-85 Hz |
| Threshold EMG level (% of MVC) | 4 |
| Required duration of threshold detection (seconds) | 2 |
| Warning time period setting (seconds) | 20 |

Referring back to FIG. 13, beginning around a time of about 285 seconds (time marker 200), elevated motor manifestations preceding the detected seizure are evident. An EMG signal may reach an amplitude value that is greater than about 4% of an amplitude value measured during a maximum voluntary contraction (MVC), which as shown in Table 2 may be the threshold amplitude for the first routine. If the EMG signal sustains a value over the threshold value for a threshold duration period of about 2 seconds, it may be deemed that the patient is exhibiting elevated motor manifestations that may be indicative of an increased risk of having a seizure. Accordingly, a positive outcome may be logged in the first routine, and a response may be initiated. For some patients, with a threshold level as low as about 4% of a maximum voluntary contraction, the first routine may log numerous positive outcomes including some that may not manifest as seizures. However, in the method of Example 2, a positive outcome in the first routine may only initiate a warning response and not an emergency response. For example, numerous positive outcomes may be made in the first routine without initiating an excessive number of costly and intrusive responses.

In Example 2, the first routine may be configured to establish a warning period of about 20 seconds during which time further signal analysis may be executed. For example, threshold detection of activity in the first routine may be made around 288 seconds. The warning period may extend for about 20 seconds ending at about 308 seconds (time marker 206). A warning message may be sent to a caregiver or otherwise logged in a database, and other routines or operations to better characterize the signal, including routines to differentiate seizure and non-seizure activity, may also be initiated. Responses that are intrusive or costly for the patient, including, for example, automatic sending of an ambulance to the patient's locale, may example, be avoided during the warning period.

A final response based on the detection of activity in the first routine may not be made until the completion of the warning period. Settings for triggering a warning period may be selected or adjusted such that some number of positive outcomes measured with the first routine may terminate without demanding active intervention by a caregiver. For example, a first routine detection may be logged and a warning message transmitted, but the warning period may terminate without resulting in an emergency alarm. For example, for some patients, settings may be adjusted so that only about 1 out of every 5 warning events or some other desired ratio of events corresponds with an actual seizure and may trigger an emergency alarm. For example, in some embodiments, a first routine threshold may be configured such that, for each positive outcome that actually manifests as a seizure, more than about 2, about 5, or about 10 non-seizure events initiate some type of warning response.

In this Example 2, a second routine may be used to monitor the collected EMG signals for the presence of qualified-clonic-phase bursts. By way of example, representative settings that may be used in a routine for qualified-clonic-phase burst analysis are shown in Table 3.

TABLE 3

| Routine Setting | Value |
|---|---|
| Frequency band selected for routine | Full Range/ |
| | 75-85 Hz |
| Threshold SNR (EMG amplitude over background) | 5 |
| Minimum clonic-phase burst duration threshold | 50 |
| (milliseconds) | |
| Maximum clonic-phase burst duration threshold | 300 |
| (milliseconds) | |
| Threshold clonic-phase burst count rate (Bursts/second) | 2 |
| Qualification routine | Individual |
| | Qualification |

As shown in Table 3, one or more frequency bands may be selected for use in the routine. For example, EMG data may be collected over one or more frequency bands, and signals in the one or more bands may be input into a detection routine.

In this Example 2, a signal amplitude may be determined for a full range including all frequencies of collected EMG signals, a signal amplitude may be determined from an isolated band ranging from about 75-85 Hz, or (as shown in Table 2) a signal amplitude may be determined using both a full range and an isolated band (e.g., ranging from about 75-85 Hz). Detection routines may be executed such as further described in detail in relation to FIGS. 6, 7, and 8. For example, with peak detection methods described therein, samples including elevated signal amplitude may be qualified by meeting a threshold SNR of about 1.25 to about 20 and by meeting a minimum threshold for duration width of about 25 to about 75 milliseconds and a maximum duration width threshold of no greater than about 250 milliseconds to about 400 milliseconds. In this Example 2, as shown in Table 3, a minimum duration threshold of about 50 milliseconds and a maximum duration threshold of about 300 milliseconds may be used. Signals that maintain an increased amplitude and which meet the aforementioned duration width thresholds may be used to characterize a patient experiencing the clonic phase of a seizure, but the thresholds may generally not be met during non-seizure periods or other parts of seizure activity. Accordingly, qualified-clonic-phase bursts may then be counted, and a number of qualified-clonic-phase bursts or a rate of qualified-clonic-phase bursts may be determined.

In some embodiments, upon processing EMG data, processed and qualified data may be written to a circular buffer in RAM in the device hardware. One advantage of such a strategy may be that less RAM may be used because the system may store only a pattern of the data (such as peak detected values and baseline noise values) and not a point by point data file of all signal data. That is, as further described in Applicant's U.S. Pat. No. 8,983,591, a voltage (or other electrical parameter that reflects EMG amplitude) at each corresponding point in time need not be stored, but only data sufficient to derive the model form may be stored.

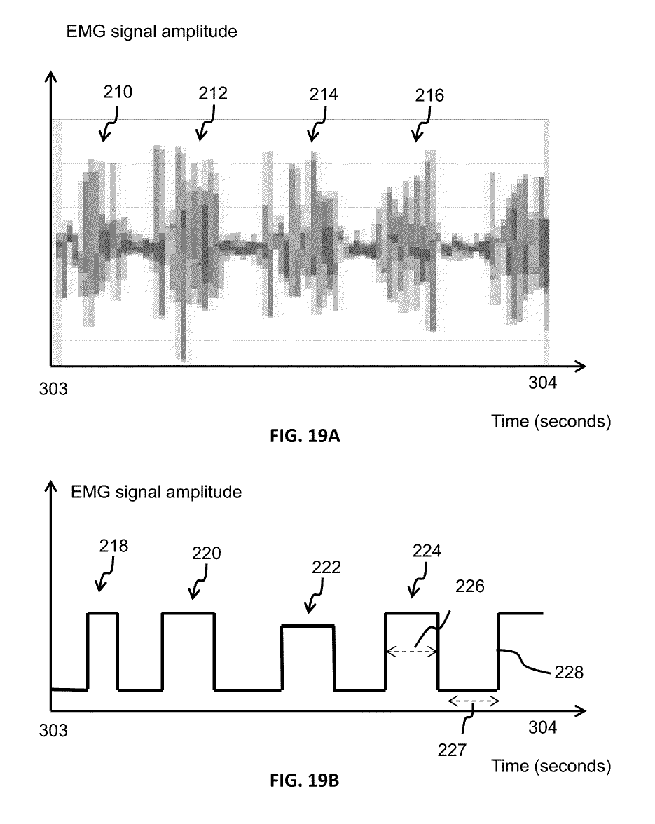

For example, a one second portion of collected EMG signal is shown in FIG. 19A. Samples (210, 212, 214, and 216) are fully present in the window of time shown in FIG. 19A. And by way of example only, in FIG. 19B, a graph showing model data that may be stored in a database based on the data in FIG. 19A is shown. Each of the samples in this Example 2 meets qualification based on conditions shown in Table 3. Therefore, each sample may be referred to as a qualified-clonic-phase burst. As shown in FIG. 19B, a model pattern for a series of 4 cleanly spaced qualified-clonic-phase bursts (218, 220, 222, and 224) may be captured and counted in the time window of FIG. 19B. Each of the qualified-clonic-phase bursts shown in FIGS. 19A and 19B includes an elevated portion and an adjacent region of reduced signal in comparison to the elevated portion. Duration widths may be compared to thresholds as shown in Table 3. For example, the clonic-phase burst 216 (also shown in FIG. 19B as model data 224) may have an associated duration width 226 for an elevated portion of the clonic-phase burst of about 110 milliseconds which is a value greater than the minimum duration width threshold (50 milliseconds) and also less than the maximum duration width threshold (300 milliseconds) shown in Table 3. In other embodiments, the duration width 227 of an adjacent quiet period next to a period of elevated signal amplitude may be included in routines configured to qualify data. In this Example 2, each time a new qualified-clonic-phase burst is qualified, an interrupt script may be triggered to automatically evaluate the data for seizure activity. For example, each time new data is qualified and written into a buffer, the method may evaluate whether an updated qualified-clonic-phase burst count may warrant a certain response.

For example, upon detection of the leading edge 228 of a next elevated portion of a sample, it may be deemed that qualified-clonic-phase burst 216 is complete and model data 224 representing the qualified-clonic-phase burst 216 may be written into a buffer. The method may then look for preceding qualified-clonic-phase bursts that may be present over a certain time period. For example, the method may look at EMG data including the qualified-clonic-phase bursts 210, 212, 214, and 216 and establish that a clonic-phase burst count of 4 is present over the time period including those clonic-phase bursts. That count rate may be above a threshold count rate which may, for example, be 2 per second (as shown in Table 3).

Referring back to the EMG data shown in FIG. 13, initial motor manifestations and/or elevated risk that the patient may be progressing to a seizure may be detected at around 288 seconds. A warning period may extend for the next 20 seconds ending near about 308 seconds (time marker 206). Prior to completion of the warning period, threshold detection of a burst count rate in the second routine may be made. An alarm may be immediately triggered based on that detection. However, in other embodiments, even if detection of clonic-phase seizure activity is made during the warning period, an alarm may not be triggered until completion of the warning period. For example, EMG data may be collected for the entirety of the warning period. Typically, where a true seizure is present, additional EMG data may be collected thereby increasing confidence that the seizure is real and only after completion of the warning period an alarm may be triggered.

### Example 3

In this Example 3, a specific embodiment of a method of analyzing collected EMG signals is described. The method may, for example, be used to detect the seizure as shown for the patient in Example 1. In the method of Example 2, a qualified-clonic-phase burst count rate was determined, and seizure activity was deemed present based on that count rate. Qualification properties were based solely on individual properties of identified samples. In contrast, in Example 3, aggregate qualification of data using a routine configured to determine the periodicity of a group of samples may be used. For example, aggregate qualification of samples may be made based on whether the samples are part of a sample train that meets qualification based on meeting of one or more percentage deviation thresholds.

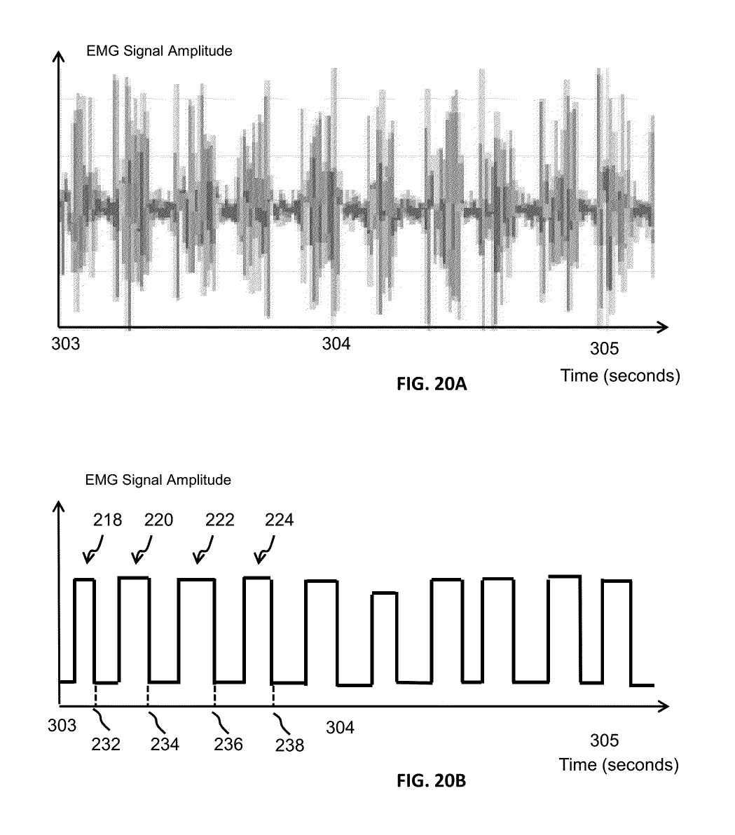

In Example 3, the periodicity of detected samples is described in reference to a series of 10 samples from the same data set shown in Example 1 and FIG. 13. The 10 samples in this example were derived from an about 2.2 second period of clonic-phase data as shown in FIG. 20A. FIG. 20B shows a model representation of that data as may be stored in one or more registers and which may be used to evaluate sample statistics. In FIG. 20B, a sample train is shown to include the 4 samples 218, 220, 222, and 224 described in Example 2 as well as other samples. The samples may meet qualification as described herein. Therefore, the samples may also be referred to as qualified-clonic-phase bursts. In addition, trailing edges (232, 234, 236, and 238) are shown for the 4 samples (218, 220, 222, and 224). The trailing edge of a sample may serve the purpose of marking a time point for the sample—although a number of other features of a sample may also serve that purpose, such as the leading edge of the sample or the sample center. In this Example 3, the samples may then be given times as follows:

303.14, 303.32, 303.56, 303.75, 304.02, 304.22, 304.46, 304.65, 304.90, and 305.08

There are 10 samples over a period of about 1.94 seconds and 9 times between the samples. The average period may be calculated as 1.94/9=0.216 seconds per sample. The time periods between samples are as follows:

303.32−303.14=0.18

303.56−303.32=0.24

303.75−303.56=0.19

304.02−303.75=0.27

304.22−304.02=0.20

304.46−304.22=0.24

304.65−304.46=0.19

304.90−304.65=0.25

305.08−304.90=0.18

The absolute values of the deviations from the average period were then calculated, and a sum of all deviations was then determined to be 0.276. The average deviation was then calculated as that sum divided by the number of periods between samples and found to be 0.0306 (0.276/9=0.0306). Or, expressed as a percentage of the average period, the average deviation percentage may be about 14.2% (0.0306/0.216×100%=14.2%). The average percentage deviation value for this train may be compared against expected values for seizure activity. For example, clonic-phase bursts may be expected to be more regular than non-seizure movements but less regular than some artificial noise artifacts. A value of 14.2% may be deemed to be well within an expected range for the periodicity calculation. For example, a minimum threshold of percentage deviation may be about 5% and a maximum threshold of percentage deviation may be about 25%. For example, when the percentage deviation is above the minimum threshold deviation, it may be expected that the samples are likely not derived from an artifact signal of a highly regular noise source such as harmonics from the power mains, fluorescent lights, etc. When the percentage deviation is less than the maximum threshold of percentage deviation of 25%, it may be expected that the samples are not from sources like non-seizure movements that tend to have greater deviation. In this Example 3, the measured percentage deviation value is within the threshold values. Accordingly, samples may be deemed to meet aggregate qualification, may be classified as qualified-clonic-phase bursts, and may be included in determining a level of qualified-clonic-phase burst activity.

In this Example 3, it does not appear that samples were missed. In the event that a low signal was manifested (or a sample happened to be present during a period of high noise), then a sample may be missed, and as opposed to a well-spaced series of samples, the spacing between adjacent suspected samples may be high. A missed sample may, if not accounted for, inordinately bias a calculated percentage deviation value. However, to lessen this bias, sample data may be tested for outliers, and if one or more individual periods in a set of periods is suspected to be an outlier, then a period may be discounted. For example, methods as described in ASTM E178 may be applied or other standard tests for detection of outlying data may be used. The filtered data (e.g., with outliers removed) may then be tested for aggregate qualification.

### Example 4

In this Example 4, a number of patients susceptible to seizures were monitored for seizure activity using EMG electrodes. Sensors were placed on the patients' biceps, EMG signals were collected, the collected signals were analyzed for the presence of seizure activity, and seizures were detected. The data in this Example 4 includes a summary of 20 different measured seizures from a total of 11 different patients. The seizures and patients herein include a subset of patients in a study to evaluate different methods for seizure detection. And, in each of the 20 seizures in this Example 4, a clonic-phase portion of the seizure was identified.

EMG data from various portions of the recorded seizures were processed to identify samples that include an elevated portion. That data was used to determine an average period between elevated portions of samples as well as duration widths of elevated portions of samples. Data was taken from each of several time spans of recorded data. For example, average duration of elevated regions of samples and average timing between elevated regions of samples were analyzed during time intervals near the start, intermediate, and later portions of the clonic phase. Table 4 shows, for a period of about 5 seconds near the start of the clonic phase, the results for the various patients and seizures in this study.

TABLE 4

| | | |
|---|---|---|
| Patient/Seizure | Time between elevated | Duration of elevated |
| Identifier | portions (sec.) | portions (sec.) |

| | | | |
|---|---|---|---|
| 1 | AcJ | 0.28 | 0.07 |
| 1 | AcJ 2 | 0.15 | 0.10 |
| 2 | FoB | 0.10 | 0.19 |
| 3 | StJ | 0.12 | 0.10 |
| 4 | LoJ | 0.22 | 0.21 |
| 5 | RiJ | 0.12 | 0.19 |
| 6 | MaL | 0.08 | 0.05 |
| 7 | MuH | 0.08 | 0.13 |
| 8 | WaA | 0.08 | 0.17 |
| 9 | PeA1 | 0.11 | 0.11 |
| 9 | PeA2 | 0.14 | 0.12 |
| 10 | McK1 | 0.14 | 0.12 |
| 10 | McK2 | 0.11 | 0.19 |
| 10 | McK3 | 0.09 | 0.10 |
| 10 | McK5 | 0.11 | 0.11 |
| 10 | McK6 | 0.07 | 0.12 |
| 10 | McK7 | 0.07 | 0.11 |
| 10 | McK8 | 0.08 | 0.07 |
| 10 | McK9 | 0.19 | 0.07 |
| 11 | ToS | 0.21 | 0.22 |

The average duration width of elevated portions of the sample at initial parts of the clonic phase for the patients in this study was about 0.12 seconds (or 120 milliseconds). By selecting threshold values for minimum burst duration width of about 25 milliseconds to about 75 milliseconds and maximum burst width threshold of no greater than about 250 milliseconds to about 400 milliseconds, samples may be identified and qualified. Thus, samples collected herein may meet qualification based, for example, on a minimum and/or maximum duration width and therefore may be referred to as qualified clonic phase bursts. Moreover, other signals that may be present during monitoring, e.g., from non-seizure sources and/or from other phases, did not follow this pattern. By selecting elevations that meet the aforementioned width requirements qualified-clonic-phase bursts may be counted and used to determine whether clonic-activity was present. By detection of clonic-phase activity the seizures may, for example, be differentiated from other seizure events that do not show clonic-phase activity.

Additional data for an intermediate portion of EMG data (about the next 5 seconds of data) is shown in Table 5. Seizure data (e.g., for times after about 10 seconds) is shown in Table 6.

TABLE 5

| | | |
|---|---|---|
| Patient and | Time between elevated | Duration of elevated |
| Recorded Seizure | portions (sec.) | portions (sec.) |

| | | | |
|---|---|---|---|
| 1 | AcJ | 0.51 | 0.15 |
| 1 | AcJS 2 | 0.20 | 0.08 |
| 2 | FoB | 0.26 | 0.18 |
| 3 | StJ | 0.18 | 0.13 |
| 4 | LoJ | 0.25 | 0.33 |
| 5 | RiJ | 0.21 | 0.18 |
| 6 | MaL | 0.17 | 0.26 |
| 7 | MuH | 0.08 | 0.14 |
| 8 | WaA | 0.20 | 0.16 |
| 9 | PeA1 | 0.15 | 0.12 |
| 9 | PeA2 | 0.17 | 0.12 |
| 10 | McK1 | 0.16 | 0.13 |
| 10 | McK2 | 0.17 | 0.17 |
| 10 | McK3 | 0.16 | 0.10 |
| 10 | McK5 | 0.21 | 0.14 |
| 10 | McK6 | 0.11 | 0.13 |
| 10 | McK7 | 0.10 | 0.10 |
| 10 | McK9 | 0.31 | 0.08 |
| 11 | ToS | 0.54 | 0.20 |

TABLE 6

| | | |
|---|---|---|
| Patient and | Time between elevated | Duration of elevated |
| Recorded Seizure | portions (sec.) | portions (sec.) |

| | | | |
|---|---|---|---|
| 1 | AcJ | 0.71 | 0.30 |
| 1 | AcJ 2 | 0.14 | 0.10 |
| 2 | FoB | 0.64 | 0.24 |
| 3 | StJ | 0.27 | 0.14 |
| 4 | LoJ | 0.49 | 0.33 |
| 5 | RiJ | 0.61 | 0.21 |
| 6 | MaL | 0.23 | 0.13 |
| 7 | MuH | 0.12 | 0.20 |
| 8 | WaA | 0.29 | 0.17 |
| 9 | PeA1 | 0.16 | 0.14 |
| 9 | PeA2 | 0.17 | 0.13 |
| 10 | McK1 | 0.39 | 0.12 |
| 10 | McK2 | 0.13 | 0.18 |
| 10 | McK3 | 0.25 | 0.11 |
| 10 | McK5 | 0.29 | 0.13 |
| 10 | McK6 | 0.14 | 0.12 |
| 10 | McK7 | 0.23 | 0.09 |
| 11 | ToS | 0.45 | 0.20 |

For some of the monitored seizures, later portions of activity were weak and/or the seizure terminated rapidly. Therefore, in some of the measured seizures, only earlier periods of activity were measured. That is, for some of the patients and/or seizures, only initial or intermediate portions were measured. Samples in any given portion of the clonic phase may be qualified and counted. For example, samples in one of the aforementioned portions of a seizure may be grouped together and tested for qualification against one or more aggregate qualification thresholds. Once qualified, various statistics for qualified-clonic-phase bursts may be determined. For example, statistics of qualified-clonic-phase bursts may be determined for the entirety of the seizure and trends throughout the seizure or through different parts of the seizure may be determined. Generally, for most patients in this study, the time between elevated regions was found to increase as the seizure progressed. For example, for the patient designated AcJ the time between elevated regions changed from about 0.28 seconds to about 0.71 seconds. Typically, for most of the patients in this study, the time between elevated regions more than doubled between the start and end of the clonic phase.

As shown in Table 7, a percentage change for times between elevated portions at the start and at the end of a seizure is shown for the patient data in this Example 4. For example, for the patient AcJ a percentage change for times between elevated portions at the start and at the end of a seizure may be calculated as:

((Time between Elevated Portions (late periods)−Time between Elevated Portions (initial periods))/Time between Elevated Portions (initial periods))×100%

((0.71 sec−0.28 sec)/0.28 sec)×100%=150%

TABLE 7

| | |
|---|---|
| | Change in times between |
| Patient and | samples for early and late |
| Recorded Seizure | periods of a seizure |

| | | |
|---|---|---|
| 1 | AcJ | 150% |
| 1 | AcJ 2 | −6% |
| 2 | FoB | 540% |
| 3 | StJ | 130% |
| 4 | LoJ | 120% |
| 5 | RiJ | 400% |
| 6 | MaL | 190% |
| 7 | MuH | 50% |
| 8 | WaA | 260% |
| 9 | PeA1 | 45% |
| 9 | PeA2 | 21% |
| 10 | McK1 | 180% |
| 10 | McK2 | 18% |
| 10 | McK3 | 180% |
| 10 | McK5 | 160% |
| 10 | McK6 | 100% |
| 10 | McK7 | 230% |
| 11 | ToS | 110% |

As shown in Table 7, for most detected events, timing between elevated regions markedly increase throughout the seizure. That information may be tracked and reported to caregivers. For example, a caregiver may search data for patterns where qualified peaks change over time in order to assist the caregiver in identifying if the patient may be prone to non-epileptic psychogenic events.

Although the disclosed method and apparatus and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. Use of the word “include,” for example, should be interpreted as the word “comprising” would be, i.e., as open-ended. As one will readily appreciate from the disclosure, processes, machines, manufacture, compositions of matter, means, methods, or steps presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

1. A method of monitoring a patient for seizure activity comprising:
monitoring said patient using one or more EMG electrodes to obtain an EMG signal;
processing with a processor said EMG signal to identify if one or more samples which include an elevated region of signal amplitude are present in said EMG signal;
determining if said one or more samples meet one or more qualification thresholds suitable to identify samples that are indicative of clonic-phase seizure activity;
classifying samples among said one or more samples that meet said one or more qualification thresholds as a first set of one or more qualified-clonic-phase bursts;
wherein said one or more qualification thresholds include one or more duration width thresholds selected from a maximum duration width threshold, a minimum duration width threshold, and a combination of both a maximum duration width threshold and a minimum duration width threshold;
including said first set of one or more qualified-clonic-phase bursts in determining a qualified-clonic-phase burst activity level;
determining if a seizure is present based on said qualified-clonic-phase burst activity level; and
initiating an alarm if said seizure is present.

2. The method of claim 1 wherein said minimum duration width threshold is a minimum duration width of an elevated region of a sample and ranges from about 25 milliseconds to about 100 milliseconds, and said maximum duration width threshold is a maximum duration width of an elevated region of a sample and ranges from about 250 milliseconds to about 400 milliseconds.

3. The method of claim 1 wherein said qualified-clonic-phase burst activity level includes a qualified-clonic-phase burst count; and
wherein said determining if said seizure is present includes comparing said qualified-clonic-phase burst count to a threshold count number.

4. The method of claim 1 wherein said qualified-clonic-phase burst activity level includes a certainty weighted qualified-clonic-phase burst value;
wherein said determining if said seizure is present includes comparing said certainty weighted qualified-clonic-phase burst value to a threshold value.

5. The method of claim 4 further comprising determining one or more statistical values for how qualified-clonic-phase bursts change throughout the course of said seizure, and providing to a caregiver a statistics summary including the one or more statistical values.

6. The method of claim 1 further comprising:
organizing said one or more samples into one or more groups;
determining one or more aggregate property values for said one or more groups;
comparing said one or more aggregate property values to one or more aggregate qualification threshold values;
determining a second set of qualified-clonic-phase bursts based on the comparing of said one or more aggregate property values to said one or more aggregate qualification threshold values; and
including said second set of qualified-clonic-phase bursts in the determining of said qualified-clonic-phase burst activity level.

7. (canceled)

8. The method of claim 6 wherein said one or more aggregate qualification threshold values are selected from a maximum deviation threshold value, a minimum deviation threshold value, and a combination thereof.

9. The method of claim 8 wherein said maximum deviation threshold value and said minimum deviation threshold value are percentage average deviation threshold values or standard deviation threshold values.

10. The method of claim 6 wherein said one or more groups include a sample number of between about 3 samples to about 20 samples.

11. The method of claim 6 wherein said one or more groups include samples identified over a time period of about 2 seconds to about 5 seconds.

12. The method of claim 6 wherein said qualified-clonic-phase burst activity level is determined from said first set of one or more-qualified-clonic-phase bursts and said second set of qualified-clonic-phase bursts by taking a union of said first set and said second set.

13. The method of claim 6 wherein said qualified-clonic-phase burst activity level is determined from said first set of one or more qualified-clonic-phase bursts and said second set of qualified-clonic-phase bursts by taking an intersection of said first set and said second set.

14. (canceled)

15. The method of claim 14 wherein said one or more statistical values are selected from a number of qualified-clonic-phase bursts, a rate of qualified-clonic-phase burst detection, an average signal-to-noise ratio of qualified-clonic-phase bursts, a spread of signal-to-noise ratio of qualified-clonic-phase bursts, an average duration width for qualified-clonic-phase bursts, a spread of a duration width for qualified-clonic-phase bursts, an average duration width of periods between elevated portions of qualified-clonic-phase bursts, a spread of duration width of periods between elevated portions of qualified-clonic-phase bursts, and combinations thereof.

16. The method of claim 14 wherein said statistical summary includes a description of trends in how qualified-clonic-phase-bursts change across one or more different portions of a clonic phase of said seizure.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method of monitoring a patient for seizure activity comprising:
monitoring the patient using one or more EMG electrodes to obtain an EMG signal;
processing with a processor said EMG signal to identify if one or more samples which include an elevated region of signal amplitude are present in said EMG signal;
organizing said one or more samples into one or more groups;
determining one or more aggregate property values for said one or more groups;
comparing said one or more aggregate property values to one or more aggregate qualification thresholds;
determining a set of qualified-clonic-phase bursts based on the comparing of said one or more aggregate property values to said one or more aggregate qualification thresholds;
including said set of qualified-clonic-phase bursts in determining a qualified-clonic-phase burst activity level;
determining if a seizure is present based on said qualified-clonic-phase burst activity level; and
initiating an alarm if said seizure is present.

42. The method of claim 41 wherein said one or more aggregate qualification thresholds are selected from a minimum deviation threshold, a maximum deviation threshold, a minimum threshold rate of sample repetition, a maximum threshold rate of sample repetition, and combinations thereof.

43. The method of claim 41 wherein said one or more aggregate qualification thresholds are selected from a maximum deviation threshold, a minimum deviation threshold, and a combination thereof.

44. The method of claim 43 wherein said maximum deviation threshold and said minimum deviation threshold are percentage average deviation thresholds or standard deviation thresholds.

45. The method of claim 41 wherein said one or more groups include a sample number of between about 3 samples to about 20 samples.

46. The method of claim 41 wherein said one or more groups include samples identified over a time period of about 2 seconds to about 5 seconds.

47. (canceled)

48. (canceled)

49. (canceled)

50. (canceled)

51. (canceled)

52. (canceled)
